SEQUENCE LISTING (4 sequences)

SEQ ID NO: 1 - protein, 4 aa, Artificial Sequence
  cathepsin B cleavage site
GFLG

SEQ ID NO: 2 - protein, 4 aa, Artificial Sequence
  lysosomal protease cleavage site
GFLG

SEQ ID NO: 3 - protein, 10 aa, Artificial Sequence
  collagenase cleavage site
GGGLGPAGGK

SEQ ID NO: 4 - protein, 7 aa, Artificial Sequence
  collagenase cleavage site
KALGQPQ

(19) United States
(12) Patent Application Publication
(10) Pub. No.: US 2015/0306181 A1
(43) Pub. Date: Oct. 29, 2015

(54) DELIVERY OF BIOTHERAPEUTICS TO THE BRAIN

(71) Applicants: Board of Regents of the University of Nebraska, Lincoln, NE (US); University of Washington Through Its Center for Commercialization, Seattle, WA (US)

(72) Inventors: Xiang Yi, Chapel Hill, NC (US); Alexander V. Kabanov, Chapel Hill, NC (US); William Banks, Lake Oswego, OR (US)

(21) Appl. No.: 14/376,969

(22) PCT Filed: Feb. 6, 2013
(86) PCT No.: PCT/US13/24915

§ 371 Date: Aug. 6, 2014

Related U.S. Application Data
(60) Provisional application No. 61/595,447, filed on Feb. 6, 2012.

(51) Int. Cl. A61K 38/22; A61K 47/48

(52) U.S. Cl. CPC A61K 38/2264; A61K 47/48215

(57) ABSTRACT

Compositions and methods for delivering therapeutic compounds to the brain are provided.

This application claims priority under 35 U.S.C. §119(e) to U.S. Provisional Patent Application No. 61/595,447, filed on Feb. 6, 2012. The foregoing applications are incorporated by reference herein.

This invention was made with government support under Grant No. NS051334-04A1 awarded by the National Institutes of Health. The government has certain rights in the invention.

### FIELD OF THE INVENTION

The present invention relates generally to formulations, particularly nasal formulations, of amphiphilic polymer conjugates and methods of use thereof. The present invention also relates to compositions and methods for the delivery of therapeutic and diagnostic agents to the brain of a patient.

### BACKGROUND OF THE INVENTION

Systemic delivery of biotherapeutics to the brain is difficult due to their poor bioavailability and limited permeation at the blood brain barrier (BBB). The administration of biotherapeutics via an intranasal route allows substances to enter the brain without exposure to peripheral clearance mechanisms or reliance on conventional BBB transport mechanisms. Traditional nasal delivery systems administer drugs in the vicinity of the turbinates and thus distribute through the systemic circulation. In contrast, intranasal to brain delivery (INB) requires drug substance to be administered into the vicinity of the cribriform plate. The pathways and mechanisms by which INB delivered substances enter the brain have been partially identified (Lochhead et al. (2012) Adv. Drug Deliv. Rev., 64:614-28). For most substances, the olfactory bulb usually has the highest uptake of any brain region after INB administration, with the other brain regions often having uptakes similar to those seen after IV administration. However, in order to improve the therapeutic outcome of the delivery of biotherapeutics to the brain, the ability to deliver the biotherapeutics to other regions of the brain is needed.

### SUMMARY OF THE INVENTION

In accordance with the instant invention, compositions and methods for inhibiting, treating, and/or preventing a disease or disorder (e.g., obesity) in a subject are provided. In a particular embodiment, the method comprises intranasally administering to the subject a composition comprising a therapeutic compound (e.g., protein) conjugated to one amphiphilic copolymer. In a particular embodiment, the therapeutic compound is leptin. The therapeutic compound may be directly linked to the amphiphilic copolymer or via a linker (cleavable or non-cleavable).

In accordance with another aspect of the instant invention, additional compositions and methods for inhibiting, treating, and/or preventing a disease or disorder (e.g., dementia) in a subject are provided. In a particular embodiment, the method comprises intranasally administering to the subject a composition comprising a therapeutic compound (e.g., protein) conjugated to more than one amphiphilic copolymer. In a particular embodiment, the therapeutic compound is leptin. The therapeutic compound may be directly linked to the amphiphilic copolymer or via a linker (cleavable or non-cleavable).

In accordance with yet another aspect of the instant invention, additional compositions and methods for inhibiting, treating, and/or preventing a disease or disorder (e.g., obesity or dementia) in a subject are provided. In a particular embodiment, the methods comprise intravenously administering to the subject a composition comprising a therapeutic compound (e.g., protein) conjugated to more than one amphiphilic block polymer. In a particular embodiment, the therapeutic compound is leptin. The therapeutic compound may be directly linked to the amphiphilic copolymer or via a linker (cleavable or non-cleavable).

### BRIEF DESCRIPTIONS OF THE DRAWING

FIG. 1 provides mass spectra of leptin (Lep)-(ss)-P85 or Lep-(ss)-L81 synthesized at pH 8.0 and pH 5.5 using different excesses of reagents.

FIG. 2A provides a schematic of the conjugation of leptin and Pluronic® P85: (I) leptin lysine modification by mono-amine Pluronic® using N-hydroxysuccinimide (NHS)-containing linker, (II) leptin N-terminal modification by mono-aldehyde Pluronic® P85. FIG. 2B provides a schematic of the synthesis of mono-propionaldehyde P85.

FIG. 3 provides the characterization of N-terminal modified leptin-(nc)-P85 samples synthesized at pH 7.4 and pH 5.5. Mass spectra of Lep-(nc)-P85 conjugates reveal unmodified leptin and leptin linked to one P85 chain (21 kDa) (FIG. 3A). Lep-(nc)-P85(1) and Lep-(nc)-P85(2) are shown to exemplify conjugates synthesized at different pH. Similar spectra were observed for all other Lep-(nc)-P85 samples. FIG. 3B shows a sodium dodecyl sulfate polyacrylamide gel electrophoresis (SDS-PAGE) of Lep-(nc)-P85 conjugates under non-reducing conditions. Native leptin was present as monomer and dimer (Lane A). All conjugates contained at least one modified monomer band (Lanes B, C, D and E).

FIG. 4 shows the purification and characterization of leptin-P85 conjugates. Elution profile from size exclusion chromatograph in TSKgel® G2000SW column showed separation of leptin-P85 conjugates from unmodified leptin (FIG. 4A). SDS-PAGE (FIG. 4B) and matrix-assisted laser desorption/ionization time of fly (MALDI-TOF) spectra (FIG. 4C) further characterized the collected conjugates to be mu-leptiPOL™-LM and mu-leptiPOL™-HM, respectively.

FIG. 5 provides hydrophobic interaction chromatography (HIC), SDS-PAGE, and MALDI-TOF analysis of Lep-(ss)-P85(2.3). FIG. 5A provides the elution profile of the native leptin and Lep-(ss)-P85(2.3). FIG. 5B provides SDS-PAGE of the native leptin (Lane A), Lep-(ss)-P85(2.3) (Lane B) and their HIC fractions eluted at 12 minutes (Lane C) and 33 minutes (Lane D). Lep-(ss)-P85(2.3) contains leptin monomer eluted 12 minutes and at least two modified forms of leptin (ca. 21 kDa, and ca. 37 kDa) both eluted at 33 minutes. FIG. 5C provides MALDI-TOF spectra of HIC fractions of native leptin and Lep-(ss)-P85(2.3) which confirm the presence of unmodified leptin eluted at 12 minutes and leptin modified with one P85 chain (21 kDa) eluted at 33 minutes.

FIG. 6 provides immunoassays of leptin-Pluronic® conjugates. FIG. 6A provides a Western blot analysis of native leptin (Lane A), Lep-(nc)-P85(2) (Lane B) and 1:1 mixture of leptin and P85 (Lane C) using anti-PEG (left panel) and anti-leptin (right panel) antibodies. FIG. 6B provides an enzyme-linked immunosorbent assay (ELISA) of native leptin, Lep-(ss)-P85(2.1), 1:1 mixture of leptin and P85, PEG-SOD1, and P85. FIG. 6C provides an ELISA of Lep-(ss)-P85(2.1), Lep-(ss)-P85(2.3) purified by HIC (33 minutes) and Lep-(ss)-L81(1).

FIG. 7 shows the characterization of the reduction of Lep-(ss)-P85(2.1) (with disulfide linker) and Lep-(cc)-P85(2.1) (with di-carbon linker) by L-glutathione by mass spectra (FIG. 7A) and ELSIA (FIG. 7B).

FIG. 8 provides surface plasma resonance (SPR) representative association and dissociation profiles. The interaction of ObR-Fc with leptin (0.3-30 nM), 1:1 mixture of leptin (0.3-30 nM) and P85, Lep-(ss)-P85(1) (10-300 nM), Lep-(ss)-P85(2.1) (5-100 nM), Lep-(nc)-P85(2) (5-100 nM) or Lep-(ss)-P85(2.3), 33min (1-30 nM) were monitored and sensorgrams were corrected by subtracting non-specific binding and baseline draft to fit to a 1:1 binding model.

FIG. 9 shows the leptin non-saturable uptake in brain and serum after INB delivery. The uptake of radioactive labeled leptin in brain olfactory bulb (OB), hypothalamus (HT), hippocampus (HC), cerebellum (CB) and overall brain (whole brain, WB) uptake as well as in serum was not inhibited by free leptin, indicating a non-saturable, leptin transporter independent mechanism for leptin to enter the brain via INB delivery.

FIG. 10 shows the brain and serum uptake of leptin and leptiPOL™ after INB delivery. FIG. 10A compares leptin levels in serum after intranasal or intravenous administration. FIG. 10B shows whole brain levels of leptin, leptiPOL™-HM, and leptiPOL™-LM after INB delivery. FIG. 10C shows whole brain/serum ratios of leptin, leptiPOL™-HM, and leptiPOL™-LM after INB delivery. FIG. 10D shows serum levels of leptin, leptiPOL™-HM, and leptiPOL™-LM after INB delivery.

FIG. 11 shows brain hypothalamus targeting by leptiPOL™-LM after INB delivery. FIGS. 11A, 11B, and 11C show the levels of leptin, leptiPOL™-HM, and leptiPOL™-LM in olfactory bulb, hypothalamus, and hippocampus, respectively. FIG. 11D provides a graph of the ratios of hypothalamus/olfactory bulb and hypothalamus/hippocampus.

FIG. 12 shows brain hippocampus targeting by leptiPOL™-HM after INB delivery.

FIG. 13 shows the effects of INB delivered mu-leptiPOL™-HM (FIG. 13A) and mu-leptiPOL™-LM (FIG. 13B) on cognition.

FIG. 14 shows food intake of mice receiving mu-leptiPOL™-LM or leptin by INB delivery.

FIG. 15 provides multiple regression analysis to calculate the influx rate of leptin-(ss)-P85 (heavy) (FIG. 15A) and leptin-(ss)-P85 (1:1) (FIG. 15B) across the BBB.

FIG. 16 shows the serum clearance of leptin-(ss)-P85 (1:1) (FIG. 16A) and leptin-(ss)-P85 (heavy) (FIG. 16B).

FIG. 17 shows the transport mechanism of the leptin-P85 conjugates through the addition of free leptin. FIG. 17A shows the transport of ¹²⁵I labeled Lep-(ss)-P85 (1:1) or ¹³¹I leptin in the presence or absence of unlabeled leptin. FIG. 17B shows the transport of ¹²⁵I labeled Lep-(ss)-P85 (heavy) or ¹³¹I leptin in the presence or absence of unlabeled leptin.

FIG. 18 shows the brain uptake of leptin conjugates (intact form).

FIG. 19 provides a schematic representation of the synthesis of poly(2-oxazoline) (POx), the conjugation of POx with SOD1, and the radiolabeling of SOD 1-POx.

FIG. 20 provides representative MALDI-ToF MS of leptin (FIG. 20A) and leptin-P(MeOx-b-BuOx) (FIG. 20B). The average molar mass and composition of each peak are labeled. (m)leptin: leptin monomer; (d) leptin: leptin dimer; (tri) leptin: leptin trimer; (tetra) leptin: leptin tetramer.

FIG. 21 provides a representative SDS-PAGE of leptin and leptin-POx. L: ladder; Leptin-POx: leptin-P(MeOx-b-BuOx).

FIG. 22 provides representative far-UV CD spectra of leptin and leptin-POx. All samples were dissolved in PBS (pH 7.4) at 0.5 mg/mL as determined by MicroBCA™ assay.

FIG. 23 shows multiple-time regression analysis of ¹²⁵I-Leptin (FIG. 23A) and ¹²⁵I-Leptin-P(McOx-b-BuOx) (FIG. 23B) transport across the BBB. ¹³¹I-albumin was co-injected in each group and the brain serum ratio of ¹³¹I-albumin was subtracted from that of ¹²⁵I-Leptin or ¹²⁵I-Leptin-P(MeOx-b-BuOx) to correct the vascular space of each individual animal. For Leptin, the unidirectional influx rate, Kⱼ=0.151±0.031 μL/g·min; vascular distribution, Vi=4.273±0.960 μ/g (r=0.80, p<0.005; n=1˜2 mice/time point). For Leptin-POx, the unidirectional influx rate, Kᵢ=0.382±0.047 μL/g·min; vascular distribution, Vi=5.203±1.407 μL/g (r=0.87, p<0.0001; n=1˜2 mice/time point).

FIG. 24 provides the brain region distribution of leptin-POx conjugates following nasal administration.

FIG. 25 shows brain hypothalamus targeting of leptin-POx (leptipox) was greatly enhanced compared to leptin relative to olfactory bulb and hippocampus following nasal administration.

### DETAILED DESCRIPTION OF THE INVENTION

Compositions and methods are provided herein for the delivery of compounds (e.g., therapeutics) to the brain (e.g., via intranasal administration). The compounds are delivered as conjugates comprising at least one polymer (e.g., amphiphilic copolymer) covalently linked (e.g., directly or via a linker) to at least one compound, particularly a polypeptide (e.g., a protein or peptide). The amphiphilic copolymer may be an amphiphilic block copolymer, e.g., one comprising at least one hydrophilic segment comprising at least one hydrophilic poly(ethylene oxide) and at least one hydrophobic segment comprising at least one hydrophobic poly(propylene oxide). In a particular embodiment, the amphiphilic block copolymer comprises at least one Pluronic®. Compositions comprising at least one conjugate and at least one carrier (e.g., a pharmaceutically acceptable carrier) are also provided. The compositions may further comprise at least one other therapeutic agent/protein and/or unconjugated amphiphilic copolymer (e.g., the unconjugated polymer may form micelles or nanoparticles). Methods of treating a disorder or disease in a subject by administering at least one conjugate or composition of the instant invention to the subject (e.g., via nasal delivery) are also provided herein.

Various kinds of gastrointestinal (GI) hormones (e.g., glucagon-like peptide 1 (GLP-1), oxyntomodulin (OXM), peptidy YY (PYY), ghrelin, pancreatic polypeptide, amylin, etc.) and adipokine such as leptin are developed as anti-obesity drugs (Colon-Gonzalez et al. (2013) Mol. Aspects Med., 34:71-83). Most of these hormones are tested in clinical trials with relatively safe profiles being reported. Most reported side effects are nausea and vomiting as often a higher dose and frequent dosing are required to reach therapeutic window for these protein-based drugs. Problems and concerns of developing these hormones arise mainly from delivery related aspect but less driven by pharmacological related toxicity. Most of these hormones signal to the central nervous system (CNS) to control the appetite. However targeting the CNS is difficulty in view of the poor bioavailability of the hormones themselves and the existence of the blood-brain barrier (BBB). Improving the pharmacokinetic profile and brain targeting is critical to advance these hormones development as anti-obesity drugs.

Currently anti-obese hormones under clinical trials are given by either subcutaneous or intravenous injection. Oral formulation of GLP-1 analog and PYY were also under development. Systemic administration of these hormones holds two problems: 1) fast clearance and degradation in blood stream and 2) inability to access its central receptor due to the hindrance of BBB. An impractical high dose level and dosing frequency are therefore needed to attain therapeutic effects. For example, combination therapy of leptin/amylin led to >10% weight loss in obese patients. In this study, leptin was dosed at 5 mg, twice daily for 20 weeks. Common injection site events and nausea were reported (Ravussin et al. (2009) Obesity 17:1736-43). Administration of substances via intranasal route allows substances to enter the brain without exposure to peripheral clearance mechanisms or reliance on conventional BBB transport mechanisms. Some peptides and proteins dramatically depart from the traditional olfactory-dominant distribution pattern and this non-classical pattern can be enhanced by modifying the peptide with cyclodextrin (Nonaka et al. (2008) J. Pharmacol. Exp. Ther., 325:513-9). This has been demonstrated with the 38 amino acid form of pituitary adenylate cyclase activating polypeptide (PACAP) (Nonaka et al. (2012) Peptides 36:168-75). Its tissues of highest uptake are the striatum and occipital cortex. Combining PACAP with beta cyclodextrin increases hypothalamic uptake by about 8-fold and hippocampal uptake by about 2-fold. This demonstrates that INB delivery can target potential therapeutics to specific brain regions.

Herein, amphiphilic block polymer was attached to the hormone leptin. The obtained conjugates were INB delivered to mice and the brain uptake, brain region distribution and efficacy in various disease models were examined. The conjugates were produced by conjugation of leptin and the amphiphilic block copolymer (e.g., Pluronic® P85) via dithiobis-(succinimidyl-propionate) (DSP). The obtained conjugates contained a mixture of unmodified leptin, leptin attached by one amphiphilic block copolymer, and leptin attached to more than one amphiphilic block copolymer. The differently modified leptins could be readily separated (e.g., by size exclusion chromatography (SEC)). The in vitro activity of these modified leptins was about 10-20 folds less than that of leptin. However, the modified leptins are taken up about 5 fold better by whole brain than native leptin. Entry into the blood stream is greater for the modified leptins as well. Overall modified leptin showed higher brain/serum ratio than that of leptin. Therefore, brain vs. periphery is relatively targeted by modified leptin, reducing peripheral off-target side effects such as immunogenicity.

Surprisingly, the uptake for the modified leptins is greater than that of leptin, not just for olfactory bulb, but also for hippocampus and hypothalamus. These regions are of particular interest as these are important sites of action for leptin's effects on appetite (hypothalamus) and cognition (hippocampus). Hypothalamic uptake relative to the olfactory bulb or hippocampal uptake is enhanced for leptin conjugated to one amphiphilic block copolymer but decreased for leptin conjugated to more than one amphiphilic block copolymer. In contrast, hippocampal uptake relative to olfactory bulb or hypothalamic uptake is enhanced leptin conjugated to more than one amphiphilic block copolymer, but decreased for leptin conjugated to one amphiphilic block copolymer. These results show that modifications with amphiphilic copolymer enhance targeting to certain brain regions.

Accordingly, the instant application has demonstrated unexpectedly that the variation of the modifications (e.g., the number of modifications) of a compound or protein will cause the compound or protein to target different regions of the brain. While the instant application has demonstrated the targeting of the hippocampus and the hypothalamus, other anatomical regions of the brain may be targeted including, without limitation: thalamus, pituitary gland, basal ganglia, cerebellum, brain stem etc. Depending on what disease or disorder is to be treated, a particular sub-region can be targeted to maximize the effect of the therapeutic agent on the desired region of the brain. For example, for Parkinson's disease, targeting to striatum in the basal ganglia region is desirable. Additionally, for brain tumors, the brain region(s) where the tumor is located and the adjacent neurons, glia cells, and vascular endothelium that develop pathological changes may be targeted. For stroke, any brain region observing pathological changes down or up signal pathway of ischemia site may be targeted.

### I. Definitions

The following definitions are provided to facilitate an understanding of the present invention:

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

As used herein, the term “lipophilic” refers to the ability to dissolve in lipids.

As used herein, the term “hydrophilic” means the ability to dissolve in water.

As used herein, the term “amphiphilic” means the ability to dissolve in both water and lipids. Typically, an amphiphilic compound comprises a hydrophilic portion and a lipophilic portion.

The term “substantially cleaved” may refer to the cleavage of the amphiphilic polymer from the protein of the conjugates of the instant invention, preferably at the linker moiety. “Substantial cleavage” occurs when at least 50% of the conjugates are cleaved, preferably at least 75% of the conjugates are cleaved, more preferably at least 90% of the conjugates are cleaved, and most preferably at least 95% of the conjugates are cleaved.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween® 80, Polysorbate 80), emulsifier, buffer (e.g., Tris HCl, acetate, phosphate), bulking substance (e.g., lactose, mannitol), excipient, auxilliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. The compositions can be incorporated into particulate preparations of polymeric compounds such as polylactic acid, polyglycolic acid, etc., or into liposomes or micelles. Such compositions may influence the physical state, stability, rate of in vivo release, and rate of in vivo clearance of components of a pharmaceutical composition of the present invention. The pharmaceutical composition of the present invention can be prepared, for example, in liquid form, or can be in dried powder form (e.g., lyophilized). Suitable pharmaceutical carriers are described in “Remington's Pharmaceutical Sciences” by E.W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “biodegradable” or “biodegradation” is defined as the conversion of materials into less complex intermediates or end products by solubilization hydrolysis under physiological conditions, or by the action of biologically formed entities which can be enzymes or other products of the organism. The term “non-degradable” refers to a chemical structure that cannot be cleaved under physiological conditions.

The term “alkyl,” as employed herein, includes both straight and branched chain hydrocarbons containing about 1 to about 20 carbons, particularly about 1 to about 15, particularly about 5 to about 15 carbons in the main chain. The hydrocarbon chain of the alkyl groups may be interrupted with heteroatoms such as oxygen, nitrogen, or sulfur atoms. Each alkyl group may optionally be substituted with substituents which include, for example, alkyl, halo (such as F, Cl, Br, I), haloalkyl (e.g., CCl₃ or CF₃), alkoxyl, alkylthio, hydroxy, methoxy, carboxyl, oxo, epoxy, alkyloxycarbonyl, alkylcarbonyloxy, amino, carbamoyl (e.g., NH₂C(═O)— or NHRC(═O)—, wherein R is an alkyl), urea (—NHCONH₂), alkylurea, aryl, ether, ester, thioester, nitrile, nitro, amide, carbonyl, carboxylate and thiol.

The term “aryl,” as employed herein, refers to monocyclic and bicyclic aromatic groups containing 6 to 10 carbons in the ring portion. Aryl groups may be optionally substituted through available carbon atoms. The aromatic ring system may include heteroatoms such as sulfur, oxygen, or nitrogen.

The singular forms “a,” “an,” and “the” include plural referents unless the context clearly dictates otherwise.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The term “treat” as used herein refers to any type of treatment that imparts a benefit to a patient afflicted with a disease or disorder, including improvement in the condition of the patient (e.g., in one or more symptoms (e.g., control appetite, improve weight (weight loss), improve cognition), delay in the progression of the condition, etc.

As used herein, the term “prevent” refers to the prophylactic treatment of a subject who is at risk of developing a condition (e.g., obesity or dementia) resulting in a decrease in the probability that the subject will develop the condition.

A “therapeutically effective amount” of a compound or a pharmaceutical composition refers to an amount effective to prevent, inhibit, or treat a particular disorder or disease and/or the symptoms thereof For example, “therapeutically effective amount” may refer to an amount sufficient to modulate obesity or dementia in a subject.

As used herein, the term “subject” refers to an animal, particularly a mammal, particularly a human.

“Dementia” generally refers to a progressive decline in cognitive function, typically due to damage or disease in the brain. “Dementia” may refer to a general mental deterioration characterized by disorientation, impaired memory, judgment, and intellect.

As used herein, the term “cognition” describes the act or process of knowing and/or high-level brain function, including awareness, judgment, concentration, focused attention, understanding and using language, learning, and/or memory.

As used herein, the term “obesity” generally refers to a condition in which there is an excess of body fat in a subject. “Obesity” may, more specifically, refer to a condition whereby a subject has a Body Mass Index (BMI; body weight per height in meters squared (kg/m²)) greater than or equal to 25.0 kg/m² , particularly greater than or equal to 30.0 kg/m².

### II. Amphiphilic Polymers

The compounds of the instant invention are conjugated/linked to one or more polymers. In a particular embodiment, the polymer comprises or consists of a hydrophilic segment or a hydrophobic segment. In a particular embodiment, the polymers are amphiphilic copolymers. The copolymer may be a random copolymer or a block copolymer, particularly amphiphilic block copolymers. Block copolymers are most simply defined as conjugates of at least two different polymer segments. Generally, amphiphilic block copolymers can be described in terms of having at least one hydrophilic (“A”) block segment and at least one hydrophobic (“B”) block segment. Thus, for example, a copolymer of the formula A-B-A is a triblock copolymer consisting of a hydrophilic block connected to a hydrophobic block connected to another hydrophilic block. Amphiphilic block copolymers which may be used in the practice of this invention include, without limitation: A-B-A, A-B, B-A-B, A-B-A-B, etc. If a main chain in the block copolymer can be defined in which one or several repeating units are linked to different polymer segments, then the copolymer has a graft architecture of, e.g., an A(B)ₙ type. More complex architectures include for example (AB)ₙ (wherein n is about 1 to about 100) or AₙBₘ, starblocks which have more than two polymer segments linked to a single center. Block copolymers structures include, without limitation, linear copolymers, star-like block copolymers, graft block copolymers, dendrimer based copolymers, and hyper-branched (e.g., at least two points of branching) block copolymers. The segments of the block copolymer may have from about 2 to about 1000, about 2 to about 300, or about 2 to about 100 repeating units or monomers. The polymers of the instant invention may comprise capping termini.

As stated hereinabove, amphiphilic block copolymers of the instant invention comprise at least one hydrophilic segment and at least one hydrophobic segment. In a particular embodiment, the hydrophilic segments are represented by polymers with aqueous solubility more that about 1% wt. at 37° C., while hydrophobic segments are represented by polymers with aqueous solubility less than about 1% wt. at 37° C. In a particular embodiment, polymers that at 1% solution in bi-distilled water have a cloud point above about 37° C., particularly above about 40° C., represent the hydrophilic segments. In a particular embodiment, polymers that at 1% solution in bi-distilled water have a cloud point below about 37° C., particularly below about 34° C., represent the hydrophobic segments. The terms “polymers represent” or “are represented by polymers” refer to polymers having the same composition and molecular mass as the segments of the block copolymer. Examples of hydrophilic segments include, without limitation, polyetherglycols, poly(ethylene oxide), methoxy-poly(ethylene glycol), copolymers of ethylene oxide and propylene oxide, polysaccharides, polyvinyl alcohol, polyvinyl pyrrolidone, polyvinyltriazole, N-oxide of polyvinylpyridine, N-(2-hydroxypropyl)methacrylamide (HPMA), polyortho esters, polyglycerols, polyacrylamide, polyoxazolines, polyacroylmorpholine, and copolymers or derivatives thereof. Examples of hydrophobic segments include, without limitation: polylactic acid (PLA), poly(lactide-co-glycolide) (PLG), poly(lactic-co-glycolic acid) (PLGA), polycaprolactone (PCL), poly(aspartic acid) (PAsp) and its derivatives, polyoxazolines, polyoxypropylene, and poly(glutamic acid) (PG1u). In a particular embodiment, the amphiphilic block copolymer comprises at least one poly(oxyethylene) segment and at least one hydrophobic segment, particularly from the list provided above or selected from the group of polylactic acid (PLA), poly(lactide-co-glycolide) (PLG), poly(lactic-co-glycolic acid) (PLGA), and polycaprolactone (PCL). In a particular embodiment, the amphiphilic block polymer is a polyoxypropylene-polyoxyethylene block copolymers (e.g., Pluronic®), poly(2-oxazoline), or polylactic acid-polyetheleneglycol block copolymer (e.g., PLA-PEG or PEG-PLA-PEG).

EO-PO polymers

In a particular embodiment, the amphiphilic block copolymer comprises at least one poly(oxyethylene) block segment and at least one poly(oxypropylene) block segment. Polyoxypropylene-polyoxyethylene block copolymers can also be designed with hydrophilic blocks comprising a random mix of ethylene oxide and propylene oxide repeating units. To maintain the hydrophilic character of the block, ethylene oxide would predominate. Similarly, the hydrophobic block can be a mixture of ethylene oxide and propylene oxide repeating units.

The polyoxypropylene-polyoxyethylene block copolymers may have the formula EOₓ-PO_{y}-EO_{z}, wherein EO: ethylene oxide units, PO: propylene oxide units, and x, y, and z are independently from about 2 to about 800, from about 5 to about 200, particularly from about 5 to about 80. The ordinarily skilled artisan will recognize that the values of x, y, and z will usually represent a statistical average of the copolymer and that the values of x and z are often, though not necessarily, the same. In a particular embodiment, x and z are independently from about 20 to about 35, particularly about 23 to about 29; and y is about 30 to about 50, particularly about 35 to about 45.

A number of such compounds are commercially available under such names as lipoloxamers, Pluronics®, Pluronic® R, poloxamers, Pluradot™, and synperonics. These block copolymers can be prepared by the methods set out, for example, in U.S. Pat. No. 2,674,619 and are commercially available from BASF under the trademark Pluronic®. Pluronic® copolymers are widely used in pharmaceutical formulation. Pre-clinical studies indicate that Pluronics® do not induce toxic effects. Indeed, no CNS-related toxicity was reported in a most recent clinical trial of doxorubicin formulated with Pluronic® (“SP1049C”) (Valle et al. (2011) Invest. New Drugs., 29:1029-37). Pluronic® block copolymers are designated by a letter prefix followed by a two or a three digit number. The letter prefixes (L, P, or F) refer to the physical form of each polymer, (liquid, paste, or flakeable solid). The numeric code defines the structural parameters of the block copolymer. The last digit of this code approximates the weight content of EO block in tens of weight percent (for example, 80% weight if the digit is 8, or 10% weight if the digit is 1). The remaining first one or two digits designate the molecular mass of the central PO block. To decipher the code, one should multiply the corresponding number by 300 to obtain the approximate molecular mass in daltons (Da). Therefore Pluronic® nomenclature provides a convenient approach to estimate the characteristics of the block copolymer in the absence of reference literature. For example, the code ‘F127’ defines the block copolymer, which is in solid flake form, has a PO block of 3600 Da (12X300) and 70% weight of EO. The precise molecular characteristics of each Pluronic® block copolymer can be obtained from the manufacturer. Examples of Pluronics® include, without limitation, L31, L35, F38, L42, L43, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R2, 17R4, 17R8, 22R4, 25R1, 25R2, 25R4, 25R5, 25R8, 31R1, 31R2, 31R4. In a particular embodiment, the amphiphilic block copolymer of the instant invention comprises P85.

Poly (2-oxazoline) Polymers

In a particular embodiment, the amphiphilic copolymer is an oxazoline copolymer. Examples of oxazoline polymers are provided in PCT/US11/31542. Poly(2-oxazoline) block copolymers of the instant invention may be synthesized by the living cationic ring-opening polymerization of 2-oxazolines. The synthetic versatility of poly(2-oxazoline)s allows for a precise control over polymer termini and hydrophilic-lipophilic balance (HLB). Block length, structure, charge, and charge distribution of poly(2-oxazoline)s may be varied. For example, the size of the hydrophilic and/hydrophobic blocks may be altered, triblock polymers may be synthesized, star-like block copolymers may be used, polymer termini may be altered, and ionic side chains and/or ionic termini may also be incorporated. Ionic side chains (e.g., comprising —R—NH₂ or R—COOH, wherein R is an alkyl) may be incorporated into the hydrophilic (preferably) or hydrophobic block. The polymers of the instant invention may also comprise units or blocks from other polymers (e.g., hybrid oxazoline polymers) such as polyethyleneoxide (PEG), polyester, polylactic acid, poly(lactide-co-glycolide), poly(lactic-co-glycolic acid), poly(acrylic acid), poly(methacrylic acid), poly(ethyleneimine), polycaprolactone, chitosan, poly(2-(N,N-dimethylamino)ethyl methacrylate), or polyamino acid (e.g. polyaspartate, poly(glutamic acid), poly(lysine) or poly(aspartic acid)).

Poly(2-oxazoline)s (also known as 2-substituted 4,5-dihydro oxazoles) are polysoaps and depending on the residue at the 2-position of the monomer can be hydrophilic (e.g., methyl, ethyl) or hydrophobic (e.g. propyl, pentyl, nonyl, phenyl, and the like) polymers. Moreover, numerous monomers introducing pending functional groups are available (Taubmann et al. (2005) Macromol. Biosci., 5:603; Cesana et al. (2006) Macromol. Chem. Phys., 207:183; Luxenhofer et al. (2006) Macromol., 39:3509; Cesana et al. (2007) Macromol. Rapid Comm., 28:608). Poly(2-oxazoline)s can be obtained by living cationic ring-opening polymerization (CROP), resulting in well-defined block copolymers and telechelic polymers of narrow polydispersities (Nuyken, et al. (1996) Macromol. Chem. Phys., 197:83; Persigehl et al. (2000) Macromol., 33:6977; Kotre et al. (2002) Macromol. Rapid Comm., 23:871; Fustin et al. (2007) Soft Matter, 3:79; Hoogenboom et al. (2007) Macromol., 40:2837). Several reports indicate that hydrophilic poly(2-oxazoline)s are essentially non-toxic and biocompatible (Goddard et al. (1989) J. Control. Release, 10:5; Woodle et al. (1994) Bioconjugate Chem., 5:493; Zalipsky et al. (1996) J. Pharm. Sci., 85:133; Lee et al. (2003) J. Control. Release, 89:437; Gaertner et al. (2007) J. Control. Release, 119:291). Using lipid triflates or pluritriflates, lipopolymers (Nuyken, et al. (1996) Macromol. Chem. Phys., 197:83; Persigehl et al. (2000) Macromol., 33:6977; Kotre et al. (2002) Macromol. Rapid Comm., 23:871; Fustin et al. (2007) Soft Matter, 3:79; Hoogenboom et al. (2007) Macromol., 40:2837; Punucker et al. (2007) Soft Matter, 3:333; Garg et al. (2007) Biophys. J., 92:1263; Punucker et al. (2007) Phys. Rev. Lett., 98:078102/1; Luedtke et al. (2005) Macromol. Biosci., 5:384; Purmcker et al. (2005) J. Am. Chem. Soc., 127:1258) or star-like poly(2-oxazoline)s are readily accessible. Additionally, various poly(2-oxazoline)s with terminal quaternary amine groups have been reported, which interact strongly with bacterial cell membranes (Waschinski et al. (2005) Macromol. Biosci., 5:149; Waschinski et al. (2005) Biomacromol., 6:235).

The copolymers of the instant invention may comprise hydrophilic and hydrophobic oxazolines. The hydrophilic and hydrophobic segments may comprise from about 1 and about 300, particularly about 5 to about 150 or about 10 to about 100 repeating units. Examples of hydrophilic 2-oxazolines include, without limitation, 2-methyl-2-oxazoline, 2-ethyl-2-oxazoline, and mixtures thereof Examples of hydrophobic 2-oxazolines include oxazolines with hydrophobic substituents (e.g., an alkyl or an aryl) at the 2-position of the oxazoline ring including, without limitation 2-butyl-2-oxazoline (including isopropyl, sec-butyl, or tert-butyl), 2-propyl-2-oxazoline (including isopropyl), and mixtures thereof In a particular embodiment, the biocompatible, water soluble polymer is a homopolymer of 2-ethyl-2-oxazoline or a copolymer (random or block) comprising 2-ethyl-2-oxazoline. In a particular embodiment, the amphiphilic block cpolymer of the instant invention comprises the formula EtOx_{A}-BuOx_{B} or MeOx_{A}-BuOx_{B}, wherein wherein A and B are independently selected between 1 and about 300, about 5 to about 150, about 10 to about 100. In a particular embodiment, A is about 40 to about 60, particularly about 45 to about 55; and B is about 10 to about 30, particularly about 15 to about 25.

### Linkers

The copolymers of the instant invention may be conjugated to the compound by any synthetically feasible site. The copolymer may be conjugated to the compound (e.g., polypeptide) directly or via a linker. For example, the linkage between the compound or protein and the copolymer can be a direct linkage between a functional group (e.g., at a termini) of the polymer and a functional group on the compound or protein. In a particular embodiment of the invention, the amphiphilic polymers are conjugated to the protein via modification of a free amino, thiol, or carboxyl group on the protein. In a particular embodiment, the amphiphilic polymers are conjugated to the protein via a disulfide bridge. The amphiphilic polymer may be mono-activated through a linker. The linker moiety may be non-biodegradable (non-cleavable) or biodegradable (cleavable). In a particular embodiment, the linker is cleaved in vivo as the conjugate either passes through the BBB or upon completion of the transfer across the BBB. In a certain embodiment of the instant invention, the linker moiety comprises amino acids that constitute a protease recognition site or other such specifically recognized enzymatic cleavage site. Exemplary protease recognition sites include, without limitation, amino acid sequences cleavable by endosomal cathepsin, such as cathepsin B (e.g., Gly-Phe-Leu-Gly (SEQ ID NO: 1); see, e.g., DeNardo et al. (2003) Clinical Cancer Res. 9:3865s-72s); sequences cleavable by lysosomal proteases (e.g., Gly-Leu-Gly and Gly-Phe-Leu-Gly (SEQ ID NO: 2); see, e.g., Guu et al. (2002) J. Biomater. Sci. Polym. Ed. 13:1135-51; Rejmanova et al. (1985) Biomaterials 6:45-48); and sequences cleavable by collagenase (e.g., GGGLGPAGGK (SEQ ID NO: 3) and KALGQPQ (SEQ ID NO: 4); see, e.g., Gobin and West (2003) Biotechnol. Prog. 19:1781-5; Kim and Healy (2003) Biomacromolecules 4:1214-23).

In another embodiment the linker region comprises a disulfide bond or hydrolysable ester. The disulfide bond may be stable in blood, but hydrolyzable by reductases present in the BBB. Representative examples of linker moieties comprising a disulfide bond include, without limitation: —OC(O)NH(CH₂)₂NHC(O)(CH₂)₂SS(CH₂)₂C(O)NH—; —OC(O)NH(CH₂)₂SS(CH₂)₂N═CH—; and —OC(O)NH(CH₂)₂SS(CH₂)₂NH—. The linker moiety may be completely cleaved or substantially cleaved, effecting the removal of the amphiphilic polymer and, optionally, most, if not all, of the linker region from the compound or protein.

Generally, the linker is a chemical moiety comprising a covalent bond or a chain of atoms that covalently attaches the therapeutic protein to the amphiphilic copolymer. The linker can be linked to any synthetically feasible position of the therapeutic protein and the polymer. In a particular embodiment the linker is attached at a position which avoids blocking the activity of the therapeutic protein.

Exemplary linkers may comprise at least one optionally substituted; saturated or unsaturated; linear, branched or cyclic alkyl group or an optionally substituted aryl group. The linker may also be a polypeptide (e.g., from about 1 to about 20 amino acids, particularly about 1 to about 10). The linker may be biodegradable under physiological environments or conditions. The linker may also be non-degradable and may be a covalent bond or any other chemical structure which cannot be substantially cleaved or cleaved at all under physiological environments or conditions. In a particular embodiment, the polypeptide is linked to the polymer via a non-degradable crosslinker (e.g., the remainder from conjugating with DSS) or a degradable crosslinker (e.g., disulfide containing linkers such as the remainder from conjugating with DSP).

### III. Proteins

While the instant invention generally describes conjugating proteins to the amphiphilic copolymers, it is also within the scope of the instant invention to conjugate other therapeutic agents or compounds of interest to the amphiphilic copolymer. Such agents or compounds include, without limitation, peptides, glycoproteins, nucleic acids, synthetic and natural drugs, lipids, small molecules, and the like.

In a particular embodiment of the instant invention, the proteins conjugated to the amphiphilic copolymers are therapeutic proteins, i.e., they effect amelioration and/or cure of a disease, disorder, pathology, and/or the symptoms associated therewith. The proteins may have therapeutic value against, without limitation, neurological degenerative disorders, stroke, Alzheimer's disease, Parkinson's disease, Huntington's disease, trauma, infections, meningitis, encephalitis, gliomas, cancers (including brain metastasis), HIV, HIV associated dementia, HIV associated neurocognitive disorders, paralysis, amyotrophic lateral sclerosis, CNS-associated cardiovascular disease, prion disease, obesity, metabolic disorders, diabetes (particularly by intravenous delivery), inflammatory disease, brain or spinal cord injury, and lysosomal diseases (such as, without limitation, Pompe disease, Niemann-Pick, Hunter syndrome (MPS II), Mucopolysaccharidosis I (MPS I), GM2-gangliosidoses, Gaucher disease, Sanfilippo syndrome (MPS IIIA), and Fabry disease). Examples of specific proteins include, without limitation, superoxide dismutase (SOD) or catalase (e.g., of mammalian, particularly human, origin), cytokines, leptin (Zhang et al. (1994) Nature, 372:425-432; Ahima et al. (1996) Nature, 382:250-252; Friedman and Halaas (1998) Nature, 395:763-770), enkephalin, growth factors (e.g., epidermal growth factor (EGF; Ferrari et al. (1990) Adv Exp Med Biol. 265:93-99), basic fibroblast growth factor (bFGF; Ferrari et al. (1991) J Neurosci Res. 30:493-497), nerve growth factor (NGF; Koliatsos et al. (1991) Ann Neurol. 30:831-840)), amyloid beta binders (e.g. antibodies), modulators of α-, β-, and/or γ-secretases, neurotrophic factors (e.g., brain-derived neurotrophic factor (BDNF) and glial-derived neutrotrophic factor (GDNF; Schapira, A.H. (2003) Neurology 61:S56-63)), vasoactive intestinal peptide (Dogrukol-Ak et al. (2003) Peptides 24:437-444), acid alpha-glucosidase (GAA; Amalfitano et al. (2001) Genet Med. 3:132-138), acid sphingomyelinase (Simonaro et al. (2002) Am J Hum Genet. 71:1413-1419), iduronate-2-sultatase (I2S; Muenzer et al. (2002) Acta Paediatr Suppl. 91:98-99), α-L-iduronidase (IDU; Wraith et al. (2004) J Pediatr. 144:581-588), β-hexosaminidase A (HexA; Wicklow et al. (2004) Am J Med Genet. 127A:158-166), acid β-glucocerebrosidase (Grabowski, G. A., (2004) J Pediatr. 144:S15-19), N-acetylgalactosamine-4-sulfatase (Auclair et al. (2003) Mol Genet Metab. 78:163-174), and a-galactosidase A (Przybylska et al. (2004) J Gene Med. 6:85-92). In a particular embodiment, the polypeptide is a GI hormone or growth factor such as, without limitation: glucagon-like peptide 1 (GLP-1), oxyntomodulin (OXM), peptidy YY (PYY), ghrelin, pancreatic polypeptide (PP), and amylin, and adipokine such as leptin. The therapeutic methods of the instant invention may comprise the administration of one or more of the therapeutic proteins (e.g., each as a conjugate with an amphiphilic copolymer) to the subject. In a particular embodiment, at least one of the polypeptides is leptin.

Leptin is a 16 kDa protein produced by fat and transported across the blood-brain barrier (BBB) into brain where it affects not just feeding, but also respiration, cognition, neurogenesis, bone density, possibly immune function, and other parameters (Zhang et al. (1994) Nature 372:425-32; Ahima et al. (1996) Nature, 382:250-252; Friedman and Halaas (1998) Nature, 395:763-770; Halaas et al. (1995) Science 269:543-6; Banks et al. (1996) Peptides 17:305-11; Schwartz et al. (1996) J. Clin. Invest., 98:1101-6). Leptin thus joins the list of hormones that are secreted peripherally but act in the CNS to exert its biological function. The short list of these hormones includes insulin and ghrelin, hormones that also have profound effects on cognition and neurogenesis, are active in models of AD, and are active after intranasal administration (Lochhead et al. (2012) Adv. Drug Deliv. Rev., 64:614-28). Leptin possess therapeutic benefits for use as a weight loss maintenance drug (Kissileff et al. (2012) Am. J. Clin. Nutr., 95:309-17).

As human and murine leptin have an 84% homology, human leptin is nearly as effective in inducing weight reduction as murine leptin in obese mice (Banks et al. (1996) Peptides 17:305-311). Human leptin (e.g., Gene ID: 3952; GenBank Accession No.: NP 000221 (e.g., a.a. 22-167)) has 8 total amino groups (Zhang et al. (1997) Nature 387:206-9), arising from the N-terminus plus 7 lysine residues (at positions 5, 11, 15, 33, 35, 53 and 94 of the mature sequence), which may be exploited to attach the copolymers of the instant invention, as described herein. Two of these amino groups (lys 11 and 15) are buried in the binding site. Lys 5, 35, 53 and 94 and the N-terminal amino group are distant from the leptin binding site (Iserentant et al. (2005) J. Cell Sci., 118:2519-27; Barrett et al. (2009) Reg. Peptides, 155:55-61). The leptin of then instant invention can be from any species, particularly human. In a particular embodiment, the leptin has at least 80%, at least 85%, at least 90%, at least 95%, or at least 99% identity with human leptin.

Leptin crosses the BBB by way of a specific, saturable transport system (Banks et al. (1996) Peptides, 17:305-311). In normal body weight animals, transport across the BBB allows leptin to access its CNS receptors. With obesity, the leptin transporter becomes increasing impaired, resulting in a resistance to circulating leptin (Banks et al. (1999) Peptides, 20:1341-1345; Banks and Farrell (2003) Am. J. Physiol. Endocrinol. Metab., 285:E10-15; Kastin et al. (1999) Peptides, 20:1449-1453; Hileman et al. (2002) Endocrinology, 143:775-783).

Several lines of evidence in humans and rodents show that impaired BBB transport is important in the maintenance and in the progression of obesity (Van Heek et al. (1997) J. Clin. Invest. 99:385-390; Banks and Farrell (2003) Am. J. Physiol. Endocrinol. Metab. 285:E10-15). In normal body weight rats and mice, in which obesity is induced with diet (that is, strains without inherent defects in leptin protein or receptor expression or downstream circuitries), leptin transporter defects predominate over brain receptor defects early on. Calculations based on CSF and serum levels of leptin indicate that in advanced obesity in humans (leptin levels of about 40 ng/ml), transporter defects account for about 2/3 of the resistance to peripheral leptin (Banks, W. A. (2003) Curr. Pharm. Des. 9:801-809). Transporter defects are acquired, reversible, and mediated only partly by excess of endogenous leptin (Banks et al. (1999) Peptides, 20:1341-1345; Banks and Farrell (2003) Am. J. Physiol. Endocrinol. Metab., 285:E10-15). Because the leptin transporter is impaired in obesity, high doses of peripherally administered leptin have to little or no effect (Heymsfield et al. (1999) JAMA, 282:1568-1575; Farooqi et al. (1999) N. Engl. J. Med., 341:879-884; Fujioka et al. (2000) NAASO Annual Meeting; Halaas et al. (1997) Proc. Natl. Acad. Sci., 94:8878-8883; Van Heek et al. (1997) J. Clin. Invest., 99:385-390; Heymsfield et al. (1999) JAMA, 282:1568-1575; Pelleymounter et al. (1998) Am. J. Physiol., 275:R950-959). Thus, the delivery of leptin into the CNS would be effective in the treatment of obesity.

As stated above, leptin is transported across the BBB by a saturable mechanism. Leptin is transported into all regions of the brain and by both the vascular and epithelial barrier, but the rate of transport, the saturation kinetics, and maximal transport vary among brain regions. The hypothalamus, which contains the arcuate nucleus, takes up the most leptin in thin and normal body weight animals, but the pons medulla and hippocampus take up the most in obese animals (Heymsfield et al. (1999) JAMA 282:1568-1575). With increasing obesity, transport of exogenously administered leptin (and the efficiency with which endogenous circulating leptin is transported) progressively decreases. Even in thin animals, the amount of leptin circulating in blood partially saturates the leptin BBB transporter (Banks et al. (2000) Am. J. Physiol. Endocrinol. Metab., 278:E1158-1165). Impaired leptin transport may be acquired. In rodents made obese with a high fat diet, the defect in transport precedes the defect in brain receptor function (Halaas et al. (1997) Proc. Natl. Acad. Sci., 94:8878-8883; Van Heek et al. (1997) J. Clin. Invest., 99:385-390). In rodents with an inborn defect in brain receptor function, the leptin transporter defect is acquired in tandem with diet-induced obesity (Levin et al. (2004) Am. J. Physiol. Regul. Integr. Comp. Physiol., 286:R143-150). In outbred obese mouse, the BBB defect is to some degree reversible with loss of body weight (Banks et al. (2003) Am. J. Physiol. Endocrinol. Metab., 285:E10-15). The defect in leptin transporter capacity is not simply caused by increased levels of circulating leptin (Banks et al. (1999) Peptides 20:1341-1345). Both obesity and starvation impair leptin transporter activity by release of triglycerides (Banks et al. (2004) Diabetes 53:1253-1260). Both endogenous and exogenous triglycerides impair leptin transport. Lowering triglycerides with pharmacologic agents enhances leptin transport. As a result of these factors, obese mice and humans are much less responsive, or even unresponsive, to peripherally administered leptin (Halaas et al. (1997) Proc. Natl. Acad. Sci., 94:8878-8883; Van Heek et al. (1997) J. Clin. Invest., 99:385-390; Heymsfield et al. (1999) JAMA 282:1568-1575; Pelleymounter et al. (1998) Am. J. Physiol., 275:R950-959). The instant invention demonstrates means by which the saturated BBB transport mechanism can be avoided and effective delivery of leptin to desired regions of the brain can be obtained.

Leptin also affects cognition and has promise as a treatment for various CNS diseases, including dementia (Banks et al. (2011) Endocrinol., 152:2539-41). A prospective study in humans with a mean follow-up of 8.3 years showed that higher plasma leptin levels were associated with a reduced hazard ratio for all cause dementia (0.68; 95% CI 0.54-0.87) and AD (0.60; 95% CI 0.46-0.79) and higher total cerebral brain volume (Lieb et al. (2009) JAMA 302:2565-72). Leptin increases hippocampal neurogenesis in vivo, acting through the leptin receptor to activate the STAT3 and PI3K-Akt pathways (Garza et al. (2008) J. Biol. Chem., 283:18238-47). In vitro studies show that leptin can reduce amyloid beta protein expression and tau phosphorylation (Greco et al. (2011) Biochem. Biophys. Res. Commun , 414:170-4; Marwarha et al. (2010) J. Alzheim. Dis., 19:1007-19). In a transgenic model of AD, leptin reduced brain and blood levels of amyloid beta protein, amyloid burden in hippocampus, and decreased phosphorylated tau (Greco et al. (2010) J. Alzheim. Dis., 19:1155-67). Leptin also reduced oxidative stress in brain in an ischemic brain injury model (Zhang et al. (2012) J. Trauma Acute Care Surg., 72:982-91). Further, 0.25-0.5 microg of leptin injected directly into the hippocampus improves learning and memory in the SAMP8 mouse (Farr et al. (2006) Peptides, 27:1420-5).

Alzheimer's disease (AD) is the most common cause of dementia in the US and much of the Western World. Currently, only two classes of drugs are approved for use, the acetylcholinesterase inhibitors and the NMDA receptor antagonist memantine; none of these drugs provide satisfactory results as assessed by health care workers or patient's families. Many other drugs successful in animal studies have failed in clinical trials (Becker et al. (2008) J. Alzheim. Dis., 15:303-25; Becker et al. (2010) Sci. Transl. Med., 2:61rv6). One of the main reasons is, surprisingly, inadequate study of PK and targeting issues, including those that relate to brain uptake and brain pharmacodynamics.

### IV. Administration of Conjugates

The amphiphilic polymer-protein conjugates described herein will generally be administered to a patient as a pharmaceutical preparation. The term “patient” as used herein refers to human or animal subjects. These amphiphilic polymer-protein conjugates may be employed therapeutically, under the guidance of a physician.

The pharmaceutical preparation comprising the amphiphilic polymer-protein conjugates of the invention may be conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of amphiphilic polymer-protein conjugates in the chosen medium will depend on the hydrophobic or hydrophilic nature of the medium, as well as the size and other properties of the amphiphilic polymer-protein conjugates. Solubility limits may be easily determined by one skilled in the art.

As used herein, “biologically acceptable medium” or “carrier” includes any and all solvents, dispersion media and the like which may be appropriate for the desired route of administration of the pharmaceutical preparation, as exemplified in the preceding discussion. The use of such media for pharmaceutically active substances is known in the art. Except insofar as any conventional media or agent is incompatible with the amphiphilic polymer-protein conjugate to be administered, its use in the pharmaceutical preparation is contemplated.

The dose and dosage regimen of an amphiphilic polymer-protein conjugate according to the invention that is suitable for administration to a particular patient may be determined by a physician considering the patient's age, sex, weight, general medical condition, and the specific condition for which the amphiphilic polymer-protein conjugate is being administered and the severity thereof. The physician may also take into account the route of administration of the amphiphilic polymer-protein conjugate, the pharmaceutical carrier with which the amphiphilic polymer-protein conjugate is to combined, and the amphiphilic polymer-protein conjugate's biological activity.

Selection of a suitable pharmaceutical preparation will also depend upon the mode of administration chosen. For example, the amphiphilic polymer-protein conjugates of the invention may be administered intravenously or intranasally. In these instances, a pharmaceutical preparation comprises the amphiphilic polymer-protein conjugates dispersed in a medium that is compatible with the site of injection.

Amphiphilic polymer-protein conjugates may be administered by any method such as, without limitation, intravenous injection or intranasal administration. Pharmaceutical preparations for injection are known in the art. If injection is selected as a method for administering the amphiphilic polymer-protein conjugates, steps must be taken to ensure that sufficient amounts of the molecules reach their target cells to exert a biological effect.

As used herein, “intranasal” administration refers to administration of a compound to the nasal cavity. The delivery can be by a spray, drops, pump, atomizer, or nebulizer or other accepted means for delivery to the nasal cavity. Particles or droplets used for intranasal administration via a spray may have a diameter (e.g., 10-500 microns) that is larger than those used for administration by inhalation to ensure retention in the nasal cavity. The compositions may be formulated as an aerosolized liquid (e.g., a nasal spray). Typically, the nasal spray will delivered a metered dose of the drug. The spray can be a manual pump or a propellant may be used. Nasal spray systems are commercially available (e.g., Becton-Dickinson Accuspray™)

Pharmaceutical compositions containing a conjugate of the present invention as the active ingredient in intimate admixture with a pharmaceutical carrier can be prepared according to conventional pharmaceutical compounding techniques. The carrier may take a wide variety of forms depending on the form of preparation desired for administration.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of amphiphilic polymer-protein conjugates may be determined by evaluating the toxicity of the molecules in animal models. Various concentrations of amphiphilic polymer-protein conjugate pharmaceutical preparations may be administered to mice, and the minimal and maximal dosages may be determined based on the beneficial results and side effects observed as a result of the treatment. Appropriate dosage unit may also be determined by assessing the efficacy of the amphiphilic polymer-protein conjugate treatment in combination with other standard drugs. The dosage units of amphiphilic polymer-protein conjugate may be determined individually or in combination with each treatment according to the effect detected.

The pharmaceutical preparation comprising the amphiphilic polymer-protein conjugates may be administered at appropriate intervals, for example, at least twice a day or more until the pathological symptoms are reduced or alleviated, after which the dosage may be reduced to a maintenance level. The appropriate interval in a particular case would normally depend on the condition of the patient.

The instant invention encompasses methods for the delivery of therapeutic agents to the hypothalamus. In particular, the instant invention encompasses methods of treating, preventing, and/or inhibiting obesity in a subject comprising the administration of at least one conjugate of the instant invention to the subject. In particular, the instant invention encompasses methods of treating, preventing, and/or inhibiting a disease or disorder (as described here, particularly those associated with the hypothalamus) comprising the administration of at least one conjugate of the instant invention to the subject. In a particular embodiment, the conjugate comprises a protein linked to one amphiphilic copolymer. In a particular embodiment, the polypeptide is a GI hormone or growth factor such as, without limitation: glucagon-like peptide 1 (GLP-1), oxyntomodulin (OXM), peptidy YY (PYY), ghrelin, pancreatic polypeptide (PP), and amylin, and adipokine such as leptin. In a particular embodiment, the polypeptide is leptin. In a particular embodiment, the conjugate is delivered intranasally. The conjugate administered to the subject may be in a composition with at least one pharmaceutically acceptable carrier. In a particular embodiment, the composition is void of or contains only trace amounts (e.g., less than about 1%) of polypeptide conjugated to more than one amphiphilic copolymer. In a particular embodiment, the composition comprises at least about 70%, at least about 80%, at least about 90%, at least about 95%, or at least about 99% polypeptide conjugated to one amphiphilic copolymer compared to free polypeptide and/or polypeptide conjugated to more than one amphiphilic copolymer. The instant methods may further comprise the step of purifying the conjugates (e.g., by size exclusion chromatography) prior to administration to the subject.

The instant invention encompasses methods for the delivery of therapeutic agents to the hippocampus. In particular, the instant invention encompasses methods of improving cognition and/or methods of treating, preventing, and/or inhibiting a disease or disorder (as described here, particularly those associated with the hippocampus) such as a neurological degenerative disorders including dementia or a dementia related disorder in a subject comprising the administration of at least one conjugate of the instant invention to the subject. Neurological degenerative disorders include, without limitation, Alzheimer's disease, Huntington's disease, Parkinson's disease, Lewy Body disease, amyotrophic lateral sclerosis, diabetic neuropathies, and prion disease. In a particular embodiment, the subject has suffered ischemic brain injury. In a particular embodiment, the disease or disorder is Alzheimer's. In a particular embodiment, the conjugate comprises a protein linked to more than one amphiphilic copolymer. In a particular embodiment, the polypeptide is a GI hormone or growth factor such as, without limitation: glucagon-like peptide 1 (GLP-1), oxyntomodulin (OXM), peptidy YY (PYY), ghrelin, pancreatic polypeptide (PP), and amylin, and adipokine such as leptin. In a particular embodiment, the polypeptide is leptin. In a particular embodiment, the conjugate is delivered intranasally. The conjugate administered to the subject may be in a composition with at least one pharmaceutically acceptable carrier. In a particular embodiment, the composition is void of or contains only trace amounts (e.g., less than about 1%) of polypeptide conjugated to one amphiphilic copolymer. In a particular embodiment, the composition comprises at least about 70%, at least about 80%, at least about 90%, at least about 95%, or at least about 99% polypeptide conjugated to more than one amphiphilic copolymer compared to free polypeptide and/or polypeptide conjugated to one amphiphilic copolymer. The instant methods may further comprise the step of purifying the conjugates (e.g., by size exclusion chromatography) prior to administration to the subject.

The above methods may also be modified for intravenous administration. As explained herein, the conjugation of multiple amphiphilic polymers to a polypeptide allows for the bypassing of saturated BBB transport mechanisms. Accordingly, the methods comprise the intravenous administration of a conjugate comprising a protein linked to more than one amphiphilic copolymer (e.g., for the treatment of dementia or obesity). In a particular embodiment, the polypeptide is a GI hormone or growth factor such as, without limitation: glucagon-like peptide 1 (GLP-1), oxyntomodulin (OXM), peptidy YY (PYY), ghrelin, pancreatic polypeptide (PP), and amylin, and adipokine such as leptin. In a particular embodiment, the polypeptide is leptin. The conjugate administered to the subject may be in a composition with at least one pharmaceutically acceptable carrier. In a particular embodiment, the composition is void of or contains only trace amounts (e.g., less than about 1%) of polypeptide conjugated to one amphiphilic copolymer. In a particular embodiment, the composition comprises at least 70%, at least about 80%, at least about 90%, at least about 95%, and/or at least about 99% polypeptide conjugated to more than one amphiphilic copolymer compared to free polypeptide or polypeptide conjugated to one amphiphilic copolymer. The instant methods may further comprise the step of purifying the conjugates (e.g., by size exclusion chromatography) prior to administration to the subject.

The following examples provide illustrative methods of practicing the instant invention, and are not intended to limit the scope of the invention in any way. While certain of the following examples specifically identify a certain type of Pluronic® block copolymer (e.g., Pluronic® P85), the use of any amphiphilic polymer is within the scope of the instant invention, as described hereinabove.

### EXAMPLE 1

### Experimental Procedures

### Materials and Methods

Mouse leptin (Lep) and mouse leptin receptor-Fc chimeras (ObR-Fc) were purchased from R&D Systems (Minneapolis, Minn.). PEG-SOD1 (S9549), 4-methoxyltrityl chloride (MTr-C1), 1,1′-carbonyldiimidazole (CDI), 1,2-ethylenediamine (EDA), ninhydrine, 3-amino-1,2-propanediol, L-glutathione, ethylenediaminetetraacetic acid (EDTA), sodium azide, ammonium sulfate, sinapinic acid, trifluoroacetic acid (TFA), triethylamine, anhydrous acetonitrile, anhydrous pyridine, methanol, dichloromethane, toluene, acetone, ethanol, isopropanol, dimethylformamide (DMF) and silica gel (288616, 70-270 mesh, 60 Å) were purchased from Sigma-Aldrich Co. (St-Louis, Mo.). Pluronic® block copolymers L81 (L81) (lot no. WSOO-25087) and Pluronic® block copolymers P85 (P85) (lot no. WPOP-587A) were obtained from BASF Corp. (Parispany, N.J.). Their characteristics are presented in Table 1. Dithiobis(succinimidyl propionate) (DSP), disuccinimidyl propionate (DSS), sodium periodate, sodium cyanoborohydride and bovine serum albumin (BSA) were from Thermo Fisher Scientific (Rockford, Ill.). Sephadex® LH-20 gel and Illustra™ NAP™-25 or -10 columns were from GE Healthcare (Piscataway, N.J.). Amicon Ultra 0.5 mL centrifugal filters (10 kDa MWCO) and Amicon Ultra centrifugal filter units Ultra-15 (MWCO 10 kDa) were from Sigma-Aldrich Co. (St-Louis, Mo.). Float-A-Lyzer® G2 (8-10 kDa MWCO) was from Spectrum Laboratories, Inc. (Rancho Dominguez, Calif.). Flexible thin-layer chromatography (TLC) plates were from Whatman Ltd (Mobile, Ala.).

TABLE 1
Structure and properties of Pluronic ® copolymers.

| Pluronic ® | Structure | M.W. | HLB ^{a} | CMC (%) ^{b} |
|---|---|---|---|---|
| L81 | EO₃-PO₄₃-EO₃ | 2750 | 2 | 0.006 |
| P85 | EO₂₆-PO₄₀-EO₂₆ | 4600 | 16 | 0.03 |

^{a} Hydrophilic-lipophilic balance.
^{b} Critical micelle concentration in aqueous solution values at 37° C. as determined using pyrene probe.

### Synthesis of Mono-amine Pluronics®

Mono-amine Pluronics® were prepared according to published methods (Yi et al. (2008) Bioconjug. Chem., 19:1071-7). Pluronic® P85 was used here as an example. Briefly, to produce mono-amine-P85, 1.2 g of P85 (M.W. 4,600) was reacted with MTr-C1 (100 mg, 1:1 molar ratio) in 15 mL of anhydrous pyridine overnight at 25° C. The reaction mixtures were purified using silica gel column (3×20 cm) and stepwise elution by 200 mL of dichloromethane containing 2%, 5% and 10% methanol. The resulting mono-MTr-P85 was isolated at 80% wt. yield. It was then activated by CDI and conjugated with EDA. Finally, the MTr protecting group was removed by treatment with TFA and mono-amine P85 was purified on gel-permeation chromatography on Sephadex® LH-20 column (2.5 x 30 cm) in methanol Amino groups were identified after TLC by color reaction with 1% ninhydrine in ethanol. No free or bis-amino-modified P85 was observed at this point in the mixture.

Conjugation of Leptin with Mono-amine Pluronics®

The modification was carried out as reported (Price et al. (2010) J. Pharmacol. Exp. Ther., 333:253-63). Again, mono-amine Pluronic® P85 was used as an example to conjugate leptin. To conjugate with the protein, the mono-amine P85 (9.3 mg) was reacted for 30 minutes at 25° C. with DSP (4.9 mg, 6-fold molar excess) or DSS (4.5 mg, 6-fold molar excess) in 0.5 mL of DMF supplemented with 0.1 mL sodium borate buffer (0.1 M, pH 8.0) and the activated copolymers were purified from the excess of reagents using Illustra™ NAP™-25 columns in 20% aqueous ethanol. About 1.5 mL of fractions containing activated copolymer were collected and immediately mixed with leptin (2 mg, molar ratio of leptin to P85 1:10, or 1:45) in 0.2 mL of sodium borate buffer (0.1 M, pH 8.0) or in 0.2 mL sodium acetate buffer (0.1 M, pH 5.5). The mixture was incubated overnight at 4° C. and then purified as described below (Lep-(ss)-P85). Similar procedures were used to produce mono-amine P81 using P81 as a starting material and then conjugate this derivative to leptin.

### Leptin Modification by Mono-Aldehyde Pluronic® Derivatives

To produce mono-aldehyde-P85, 1 g of mono-MTr-P85 was activated for 2 hours at 25° C. with CDI (50 mg, 5-fold molar excess) in 10 mL of anhydrous acetonitrile and then reacted overnight at 25° C. with 3-amino-1,2-propanediol (0.16 mL, 10-fold molar excess) in 20 mL of ethanol. The excess of the reagents was removed on Sephadex® LH-20 column (2.5×30 cm) in methanol. The MTr protecting group was removed by a 1 hour incubation of the copolymer in 50 mL of 2% TFA in dichloromethane at 25° C., followed by neutralization of the acid with 5 mL of 10% triethylamine in methanol and purification on Sephadex® LH-20 column (2.5×30 cm). The 3-amino-1,2-propanediol-derivative of P85 was dried in vacuo and stored at -20° C. It was used to generate mono-aldehyde-P85 immediately prior to conjugation with the protein.

For conjugation with leptin, 3-amino-1,2-propanediol-derivative of P85 (20 mg) was oxidized by sodium periodate (5 mg, 5-fold molar excess) in 1 mL of methanol supplemented by 100 uL of sodium acetate buffer (0.02 M, pH 5.5) in the dark for 0.5 hour at 4° C. Excess of the oxidant was removed on Illustra™ NAP™-25 column in 20% aqueous ethanol. The resulting mono-aldehyde-P85 (10 mg) was immediately reacted with leptin (1 mg, molar ration of leptin to polymer, 1:30) in 1.5 mL of 20% aqueous ethanol supplemented by 200 uL of sodium acetate (0.1 M, pH 5.5) or sodium phosphate (0.1 M, pH 7.4) buffer. The reaction was allowed to proceed for 3 hours at 25° C. and another 12 hours at 4° C. after adding sodium cyanoborohydride (15 mM). The reaction was then terminated by adding excess of triethyleneamine for 1 hour at 25° C. These leptin-P85 conjugates (Lep-(nc)-P85) were purified on the Illustra™ NAP™-25 column in 20% aqueous ethanol and precipitated in cold acetone to remove any non-conjugated copolymer.

### N-terminal Sequencing

N-terminal sequencing was conducted by regular Edman degradation using ABI Procise® 494 Sequencer. Briefly, 20 pmol of leptin or Lep-(nc)-P85 conjugates (Lep-(nc)-P85(1) and (2)) was reacted with the Edman reagent. The first five cleaved N-terminal amino acids were analyzed by HPLC. The portion of N-terminus modified leptin in Lep-(nc)-P85 samples was determined by a decrease in the portion of the N-terminal amino acid susceptible to Edman degradation.

### Purification of Leptin-Pluronic® Conjugates

Leptin-Pluronic® conjugates were further purified in order to remove excess of free Pluronic® polymer and/or non-modified protein. The non-reacted copolymers were removed by precipitating the reaction product, Lep-(ss)-P85 or Lep-(nc)-P85 in cold acetone. The obtained precipitates were subjected to size exclusion chromatography (SEC) on TSKgel® G2000SW column (7 8 mm×30 cm, Tosoh Bioscience LLC, Grove City, Ohio) in Shimadzu HPLC system with a multiple-wavelength UV-detector (Shimadzu Scientific Instruments, Columbia, Md.) to separate unmodified leptin, leptin attached with 1 Pluronic®. Protein fractions were eluted in 0.1 M Na₃PO₄/0.2 M NaCl (pH 7.4) containing 5% methanol at flow rate of 1 mL/min and then desalted using Amicon Ultra centrifuge filter unit. The conjugates were then analyzed by mass spectra and SDS-PAGE stained by SYBRO® Ruby solution (Sigma-Aldrich, St. Louis, Mo.). Alternatively, the samples were purified without acetone precipitation by directly subjecting to hydrophobic interaction chromatography (HIC) on TSKgel® Phenyl-5PW column (7 5 mm×75 cm, Tosoh Bioscience LLC) in Agilent HPLC system 1200 (Agilent Tech., Foster City, Calif.) using as eluents (A) 1 M ammonium sulfate, 0.05 mM sodium phosphate, pH 7.0 and (B) 0.05 mM sodium phosphate, pH 7.0, 25% isopropanol (linear gradient 20% to 60% B over 10 minutes, then to 100% of B over 20 minutes, then 100% B for 15 min) at the flow rate 1.0 mL/min. The fractions were dialyzed against deionized water at 4° C. and lyophilized.

### MALDI-TOF Spectra

Mass values of leptin-Pluronic® conjugates were determined by matrix-assisted laser desorption/ionization time of fly (MALDI-TOF) spectroscopy in 4800 MALDI TOF/TOF™ analyzer (Applied Biosystems/MDS SCIEX), at a laser power of 3000 V and in positive reflector mode. Solution containing saturated sinapinic acid in 50% acetonitrile with 0.1% TFA was used as matrix for sample preparation. Briefly, 0.5 μL of sinapinic acid solution was coated on the plate followed by 1) depositing 0.5 μL solution of salt free leptin-Pluronic® conjugates in water (10-4 M), and, 2) coating with 0.5 μL sinapinic acid solution. The mass spectrometer was calibrated against insulin (5729.61 Da) and albumin (66429.09 Da) (Sigma-Aldrich Co. St-Louis, Mo.).

### SDS-PAGE Assay

Sodium dodecyl sulfate polyacrylamide gel electrophoresis (SDS-PAGE) and Western blot were performed. Leptin, leptin-Pluronic® conjugates, mixture of leptin and P85 (4:1 by weight), and other reference samples were prepared in 5 μL deionized water at a protein concentration of 2 μg/82 L (as determined by MicroBCA™M) and diluted (1:1) with non-reducing denaturing loading buffer (3.8 mL of H₂O, 5 mL of 0.5 M Tris HCl (pH 6.8), 8 mL 15% w/v SDS, 4 mL of glycerol, 0.4 mL of bromophenol blue 1% w/v). The samples were heated for 5 minutes at 100° C. and then loaded to 15% precast polyacrylamide Tirs-HCl gel (Bio-Rad Life Science Res., Hercules, Calif.). After running for 3 hours at 120 V, the gel was fixed in 50% methanol/10% acetic acid, stained in SYPRO® Ruby solution and scanned on a Typhoon gel scanner. Western blot was conducted by transferring the non-stained gels to nitrocellulose paper in Tris/Glycine transfer buffer (Bio-Rad) overnight at 20 V. Blots were then blocked for 2 hours with 10% skim milk/0.02% BSA in PBS-T (0.05% Tween® 20 in PBS), washed thrice in PBS-T and then incubated for 2 hours at 25° C. with solutions containing 0.02% BSA and either 0.2 μg/mL anti-leptin antibody (AF498, goat IgG anti-mouse leptin, R&D Systems, Minneapolis, Minn.) or 5 μg/mL anti-PEG antibody (AGP4, mouse IgM anti-PEG, Academia Sinica, Taibei, Taiwan) in PBS-T. After that the blots were washed again (thrice with PBS-T and twice with PBS) and treated with solutions containing 0.02% BSA and either rabbit anti-goat Ig-HRP (10,000 times dilution, from Sigma-Aldrich Co., St-Louis, Mo.) in PBS-T for leptin detection or donkey anti-mouse IgM-HRP (50,000 times dilution, from Jackson ImmunoResearch Lab, West Grove, Pa.) in PBS-T for Pluronic® detection. After 1 hour incubation at 25° C., the blots were washed thrice with PBS-T and twice with PBS and visualized using ECL detection kit (Thermo Fisher Scientific., Rockford, Ill.) according to the manufacturer's protocol.

### Enzyme-Linked Immunosorbent Assay (ELISA)

The ELISA protocol by Academia Sinica (Taipei, Taiwan) was followed. Briefly, 96-well microplates (eBioscience, Inc., San Diego, Calif.) were coated first for 4 hours at 37° C. and then overnight at 4° C. with 50 μL/well of AGP4 antibody (5 μg/mL) in 35 mM NaHCO₃, 15 mM Na₂CO₃, pH 9.3, then blocked with 5% skim milk in PBS for 2 hours, and washed thrice with PBS. The analyzed samples in 50 μL dilution buffer (2% skim milk in PBS) were added to each well and incubated 2 hr at 25° C. Plates were washed (thrice with PBS-T and twice with PBS) and supplemented with 50 μL/well biotinylated anti-PEG antibody (3.3-biotin, 5 μg/mL in dilution buffer, Academia Sinica, Taipei, Taiwan). After 1 hour at 25° C. the plates were washed and stained for 1 hour with 50 μL/well of streptavidin-HRP (1 μg/mL, Jackson ImmunoResearch Lab, West Grove, Pa.). Finally, the plates were washed again and peroxidase activity was measured by adding 100 μL/well tetramethylbenzidine (Thermo Fisher Scientific, Rockford, Ill.) for 5-30 minutes followed by 100 μL/well of stopping reagent (Thermo Fisher Scientific, Rockford, Ill.). Absorbance (450 nm) was measured in microplate reader SpectraMax® M5 (Molecular devices, Sunnyvale, Calif.).

### Size Measurement

Particle size and size distribution were measured by dynamic light-scattering (DLS) using Zetasizer Nano-ZS instrument (Malvern, UK). Samples were prepared at 100 μg/mL concentration in deionized water, sterilized by 0.22 μm of sterile Ultrafree-MC centrifugal filter units and kept at equilibrium at 20° C. for 5 minutes prior to measurement. The particle parameters were measured for 15 minutes at 25° C. with a 90° scattering angle. Mean effective hydrodynamic diameter (Deff) and number-average size distribution were obtained by automatically repeating (six times) the measurement based on the Zetasizer internal setting.

### Disulfide Bond Reduction

The reduction of the disulfide bond in the linker of the leptin-Pluronic® conjugates was performed in the presence of physiological intracellular concentration of L-glutathione. Briefly, Lep-(ss)-P85(1) and Lep-(ss)-P85(2.1) or control samples of Lep-(cc)-P85(1) and (2.1) (50 μg in 500 uL PBS, pH 7.4) were dialyzed in Float-A-Lyzer® (8-10 KDa) against PBS containing 20% ethanol and 6 mM reduced L-Glutathione at 4° C. overnight (12-20 hours) and with three complete buffer changes. Samples were then purified using Amicon Ultra 0.5 mL centrifugal filters to remove excess of reducing reagent. Protein content was measured using reducing agent compatible BCA Protein Assay (Thermo Fisher Scientific, Rockford, Ill.) and characterized by mass spectra and ELISA.

### Binding Affinity Measurement

The binding affinity of leptin and its analogs was measured by surface plasma resonance (SPR) in Biacore® 3000 instrument (GE Healthcare, Piscataway, N.J.), using a method reported for leptin (Mistrik et al. (2004) Anal. Biochem., 327:271-7). Carboxymethyl dextran chip (CMS), N-hydroxysuccinimide (NHS), 1-ethyl-3-(3-dimethylaminopropyl) carbodiimide (EDC), ethanolamine-HCl, HBS-EP buffer (10 mM HEPES, 150 mM NaCl, 3 mM EDTA, 0.005% surfactant P20, pH 7.4) and protein G were all purchased from GE Healthcare. To prepare the sensor chip, protein G was immobilized on CMS (Channel A and B) by consequent injection of 1) 115 μL NHS/EDC (1:1, v/v) (to activate dextran surface); 2) 60 μL protein G (200 μg/mL, 10 mM sodium acetate buffer, pH 4.0) (to bound the surface at 2000-3000 resonance units (RU)), and 3) 75 μl of 1 M ethanolamine hydrochloride, pH 8.5 (to deactivate NHS-ester and remove electrostatically bound protein). CMS surface response was recorded by first, immobilization of ObR-Fc (0.5 μg/mL, 15 μL in HBS-EP, 5 μL/min flow rate) in channel A following 1800 s washing (to capture 100-300 RU of ObR-Fc); second, capture of leptin or leptin-Pluronic® conjugates (0-300 nM, 100 μL in HBS-EP, flow rate 20 μL/min) and dissociation (900 s) in channel A (ObR-Fc surface) and B (protein G surface); third, regeneration of a fully active protein G surface by 5 μL glycine (10 mM, pH 2.0). The data (sensorgrams in channel A) were corrected by non-specific protein G surface binding (sensorgrams in channel B) and baseline draft (sensorgrams of HBS-EP injection in channel A) which might occur due to a slow dissociation of between ObR-Fc and protein G and then fitted to a 1:1 binding model using BIA evaluation software.

### Animal Studies

In vivo experiments were conducted in CD-1 male mice (8 to 10 weeks of age) (Charles River Laboratories, Wilmington, Mass). The mice had free access to food and water and were maintained on a 12-hour dark/light cycle in a room with controlled temperature (24±1° C.) and humidity (55±5%). These experiments were conducted in Geriatrics Research Education and Clinical Center, Veterans Affairs Puget Sound Health Care System and Division of Gerontology and Geriatric Medicine, Department of Internal Medicine, University of Washington. All the procedures are approved by the National Institutes of Health Guide for Care and Use of Laboratory Animals.

### Radioactivity Labeling

Leptin or leptin-P85 conjugates were radioactively labeled with ¹²⁵I (PerkinElmer Life and Analytical Sciences, Boston, Mass.) using chloramine-T method. Briefly, 5 μg of leptin (1 μg/μL in 5 uL ddH₂O) or 10 μg of leptin conjugates (1 μg/μL in 10 μL ddH₂O) was mixed with 0.5 mCi ¹²⁵I to final volume of 35 μL in chloride free sodium phosphate buffer (0.25 M, pH 7.5). The mixture was incubated with 10 μg of chloramine-T solution (2 μg/μL, freshly made in 5 μL sodium phosphate buffer (0.25 M, pH 7.5)) with vortex. The reaction was stopped exactly after 60 seconds by adding 10 μg of sodium metabisulfite solution (2 μg/μL, freshly made in 5 μL sodium phosphate buffer (0.25M, pH 7.5)). The mixture was then loaded to Sephadex® G-10 column (home-made in 2 mL glass pipet) and the ¹²⁵I labeled materials were collected in eppendorf tube pretreated with 100 μL of lactated Ringer's solution (LR) with 1% BSA (%1 BSA-LR). 10 μL of collected ¹²⁵I labeled material was added to 0.5 mL of 1% BSA-LR and then precipitated in 0.5 mL of 30% trichloroacetic acid (TCA) followed by centrifuging at 5400 g for 10 minutes at 4° C. The resulting supernatant and pellet were counted in a PerkinElmer γ-counter and used to calculate ¹²⁵I association based on the percentage of the radioactivity of the pellet among the total radioactivity of the pellet and the supernatant. More than 98% of association was observed for both ¹²⁵I labeled leptin and leptin-P85 conjugates. Similarly albumin was labeled by ¹³¹I using the method described above.

### Intranasal Delivery and Brain Pharmacokientics

Male CD-1 mice (2 month old) purchased from Charles River were anesthetized with an i.p. injection of 0.2 ml of urethane (40% solution) and the right carotid artery is exposed. Mice are given bilaterally an intranasal (INB) administration of 2 μl of lactated Ringer's solution (LR) containing 1% bovine serum albumin (BSA) and 500,000 cpm/μl of leptin or a leptiPOL™. The 2 μl is delivered to the cribriform plate by pushing a small cannula attached to a 10 μl syringe through the right and left nares (total: 4 μl/mouse) to the depth of the cribriform plate. Blood is collected from the right carotid artery and the whole brain removed at 5, 10 and 20 minutes (n=5 mice/time) after the INB administration. The brain is dissected after the method of Glowinski and Iversen on ice into the olfactory bulb, cortex, hippocampus, hypothalamus, cerebellum and remaining brain, the regions weighed, and their radioactivity levels measured in a gamma counter. Whole blood is centrifuged at 5,400 g for 15 minutes at 4° C. and the radioactivity measured in 50 μl of serum. Values are calculated and analyzed.

### Pharmacokinetics Data Analysis

The results were interpreted as brain region/serum ratios and as percent of the administered dose present in each g of brain region tissue. Each region including the whole brain was statistically compared to leptin by two-way analysis of variance (ANOVA) and if an overall difference was found then a Newman-Keuls post-test will be performed to discover where those differences lie. The compound and time are the independent variables and % Inj/g, % Inj/ml, or brain/serum ratios are the dependent variables. The observations in these experiments are independent.

The main analysis was based on the percent of the intranasal dose that was taken up per g of whole brain or the brain region of interest (% Inj/g). Whole brain values were calculated by summing the levels of radioactivity and weights for hippocampus, hypothalamus, and remainder of brain. Uptake in blood was measured by calculating the percent of the INB dose that appears in a ml of serum (% Inj/ml). Brain/serum ratios were calculated only for whole brain: Brain/serum ratio (microl/g)=1000(Rcpm)/[(Rwt)(Scpm)].

### Cognition Test in Alzheimer's Disease Mouse Model

The SAMP8 is a natural mutation that with aging develops an amyloid beta protein dependent impairment in learning and memory (Flood et al. (1998) Neurosci. Biobehay. Rev., 22:1-20; Morley et al. (2002) Peptides 23:589-99). Defects in learning and memory are widespread in the SAMP8 as demonstrated by impairments in the active-avoidance T-maze, the passive-avoidance T-maze, the Morris water maze, object recognition, the Barnes maze, and lever press (Erickson et al. (2012) J. Alzheim. Dis., 28:951-60; Farr et al. (2003) J. Neurochem., 84:1173-83; Banks et al. (2011) J. Alzheimer's Dis., 23:599-605; Farr et al. (2012) J. Alzheimer's Dis., 28:81-92; Sandoval et al. (2012) Eur. J. Pharmacol., 683:116-24). The aged SAMP8 has many findings reminiscent of AD: increased brain levels of amyloid beta protein, oxidative stress of brain proteins and membranes, cholinergic deficits, and impairments in brain efflux systems such as LRP-1, p-glycoprotein, and CSF reabsorption (Farr et al. (2003) J. Neurochem., 84:1173-83; Flood et al., Age-related changes in the pharmacological improvement of retention in SAMP8 mice. In: Takeda, T., ed. The SAM Model of Senescence. Kyoto:Excerpta Medica; 1994, p. 89-94; Farr et al. (2003) Life Sci., 73:555-62; Poon et al. (2004) Neurosci., 126:915-26; Petursdottir et al. (2007) Neurobiol. Aging, 28:1170-8; Farr et al. (2000) Neurobiol. Learning Memory, 73:150-67). SAMP8 do not have obvious plaques as in AD, probably because mouse amyloid beta peptide aggregates less than human amyloid beta peptide, but amorphous plaques are demonstrable by about 18 mo of age (Morley et al. (2000) Peptides, 21:1761-7; Akiyama et al. (1986) Acta Neuropathol., 72:124-9; Takemura et al. (1993) Amer. J. Pathol., 142:1887-97). All of these features are reversed when aged SAMP8 mice are treated with antisense directed against amyloid precursor protein (APP) or by passive immunization with antibodies directed against amyloid beta peptide (Poon et al. (2004) Brain Res., 1018:86-96; Erickson et al. (2012) J. Alzheimer's Dis., 28:951-60; Farr et al. (2003) Life Sci., 73:555-62; Kumar et al. (2000) Peptides, 21:1769-75; Banks et al. (2001) J. Pharmacol. Exper. Ther., 297:1113-21; Morley et al. (2002) Neurobiol. Learning Memory, 78:125-38; Banks et al. (2005) Peptides, 26:287-94; Banks et al. (2007) Exper. Neurol., 206:248-56). Additionally, oxidative stress both for whole brain and for specific brain proteins is increased in the aged SAMP8. Both oxidative stress and cognition improve in aged SAMP8 mice when they are treated with either antioxidants or with APP antisense (Poon et al. (2004) Brain Res., 1018:86-96; Farr et al. (2003) J. Neurochem., 84:1173-83; Poon et al. (2004) Neurosci., 126:915-26; Petursdottir et al. (2007) Neurobiol. Aging, 28:1170-8).

The cognitive function of leptiPOL™ treatment was tested in this nontransgenic SAMP8 mouse model of AD. 12 month old SAMP8 mice are habituated for 3 days to the testing apparatus. On the first day of training, mice are placed in thetesting apparatus for 5 minutes and allowed to explore a pair of identical objects, then anesthetized and given INB saline, leptin, or a leptiPOL™ either 15 minutes prior to training (short-term retention test) or immediately after training (long-term retention test). Either 5 minutes or 24 hours later, one of the original objects is replaced with a new, novel object. The amount of time a mouse spends investigating the novel object is recorded. An equal time spent with both objects indicates no recollection of the original object, whereas less than 50% of time spent with the original object indicates a recollection of the object.

Active Avoidance T-maze is a complex reference-memory task shown to test hippocampal-dependent memory including effects of leptin (During et al. (2003) Nat. Med. 9:1173-1179; Banks et al. (2004) J. Pharm. Exp. Thera. 309:469-475). A cue buzzer sounded at 55 dB, 5 seconds before a foot-shock set at 0.35 mA is applied, is used with a 35 second inter-trial interval. Immediately after training, the anesthetized mouse is given INB saline, leptin, or a leptiPOL™. Retention is tested one week later by continuing the training until mice reach the criterion of 5 avoidances in 6 consecutive trials.

### Feeding Study

CD-1 Mice are food deprived overnight by removing all food at 5 PM the night before study. Fifteen minutes after INB, the mouse is weighed placed in a cage containing a weighed food pellet. The pellet is weighed every 30 minutes for 4 hours. The pellet and mouse are again weighed 24 hours after the feeding session began. The amount of food eaten is calculated for the 30 minutes, lhour, 4 hours, and 24 hours after leptin or leptiPOL™ injection and/or the change in body weight over 24 hours calculated.

### Results

Synthesis and Characterization of Leptin-Pluronic® Conjugates

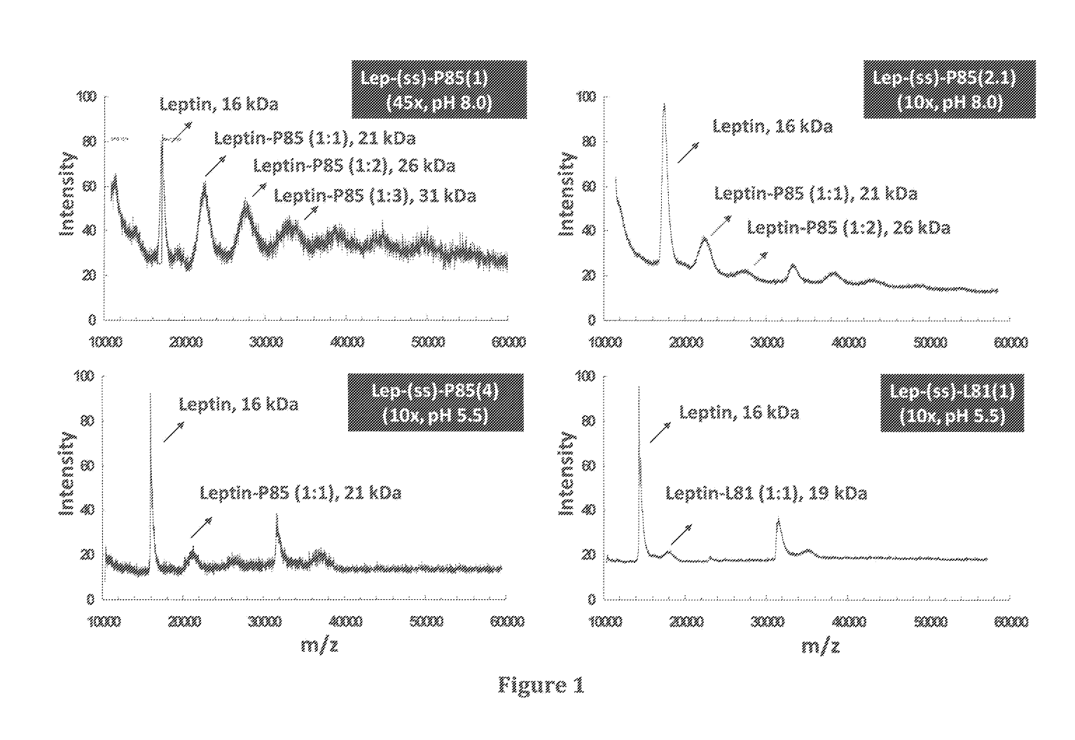

Two methods were developed to covalently attach Pluronic® block copolymers to leptin. The first method was a modification of lysine amino groups of leptin by mono-amine Pluronic® using N-hydroxysuccinimide (NHS)-containing homo-bifunctional linking agents (degradable DSP and non-degradable DSS). This procedure has been described for modification of HRP, SOD 1 and leptin, but with DSP only. The linker molecules were used to activate mono-amine Pluronic® P85 or L81 derivatives, which were then reacted with leptin in 20% ethanol in sodium borate buffer (pH 8.0) or sodium acetate buffer (pH 5.5) (Table 2). The reactions proceeded readily and generated leptin-Pluronic® conjugates linked through degradable (Lep-(ss)-P85) or non-degradable (Lep-(cc)-P85, Lep-(cc)-L81) groups. All samples contained mixtures of unmodified leptin and leptin modified with 1 to several polymer chains, as determined by mass spectra (FIG. 1). Notably, at a 45 fold molar excess of copolymer and using alkaline conditions, a highly modified sample Lep-(ss)-P85(1) was obtained. The pharmacokinetics and food intake control properties of this analog in animal models have been reported (Price et al. (2010) J. Pharmacol. Exp. Ther., 333:253-63).

TABLE 2
Conjugates of leptin with Pluronic ® P85 or L81.

| | | Pluronic ®:Leptin | Reaction | Purification |
|---|---|---|---|---|
| Conjugate | Linker | molar ratio | pH ^{a} | method ^{b} |
| Lep-(ss)- | DSP | 45 | 8.0 | A |
| P85(1)^{d} | | | | |
| Lep-(ss)- | DSP | 10 | 8.0 | A |
| P85(2.1) | | | | |
| Lep-(ss)- | DSP | 10 | 8.0 | A and SEC |
| P85(2.2) | | | | |
| Lep-(ss)- | DSP | 10 | 8.0 | HIC |
| P85(2.3) | | | | |
| Lep-(ss)- | DSP | 10 | 5.5 | A |
| P85(3) | | | | |
| Lep-(cc)- | DSS | 45 | 8.0 | A |
| P85(1) | | | | |
| Lep-(cc)- | DSS | 10 | 8.0 | A |
| P85(2.1) | | | | |
| Lep-(ss)- | DSP | 10 | 5.5 | A |
| L81(1) | | | | |
| Lep-(nc)- | NA^{c} | 30 | 7.4 | A |
| P85(1) | | | | |
| Lep-(nc)- | NA^{c} | 30 | 5.5 | A |
| P85(2) | | | | |
| Lep-(nc)- | NA^{c} | 10 | 5.5 | A |
| P85(3) | | | | |
| Lep-(nc)- | NA^{c} | 60 | 5.5 | A |
| P85(4) | | | | |

^{a} Last stage of conjugation of leptin with activated copolymer derivative was carried out at different pH using either 0.1M sodium borate buffer (pH 8.0) or 0.1M sodium acetate buffer (pH 5.5).
^{b} The excess of Pluronic ® was removed by acetone precipitation (A); HPLC of size exclusion chromatography (SEC), or HPLC of hydrophobic interaction chromatography (HIC).
^{c}NA: not applicable. Lep-(nc)-P85 was generated by reductive amination of leptin N-terminal α amine group with mono-aldehyde-P85 in the presence of sodium cyanoborohydride.
^{d}The in vivo studies of this sample was previously reported.

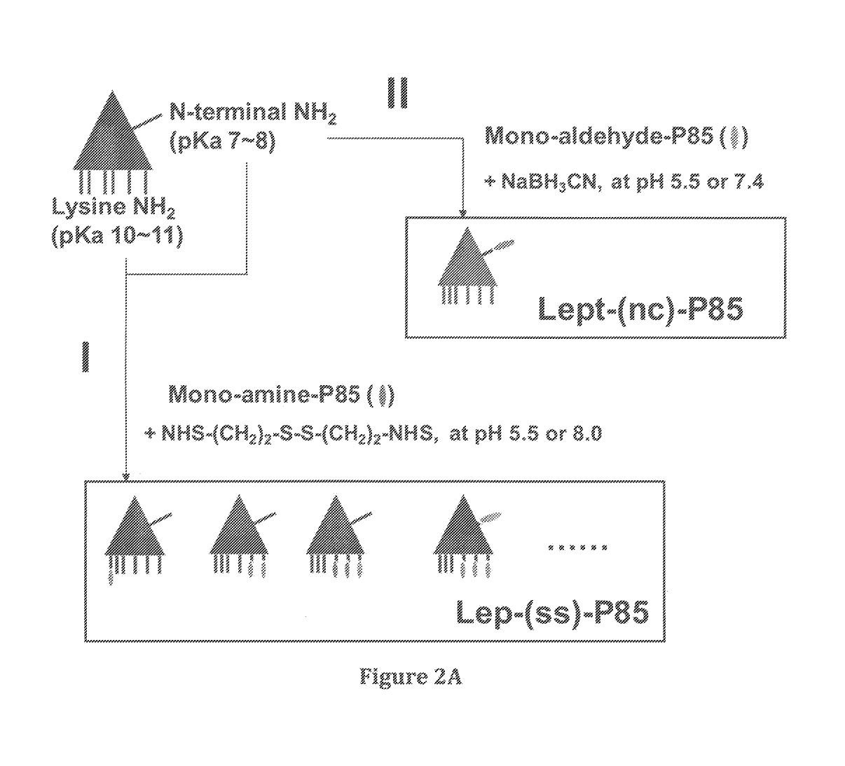

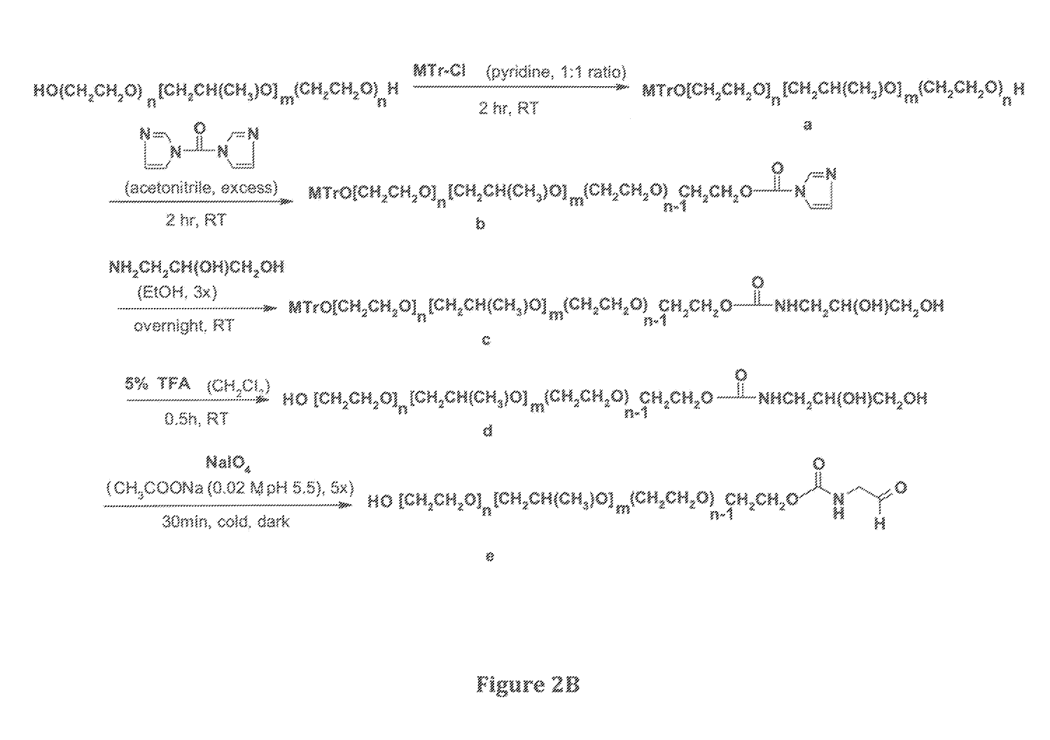

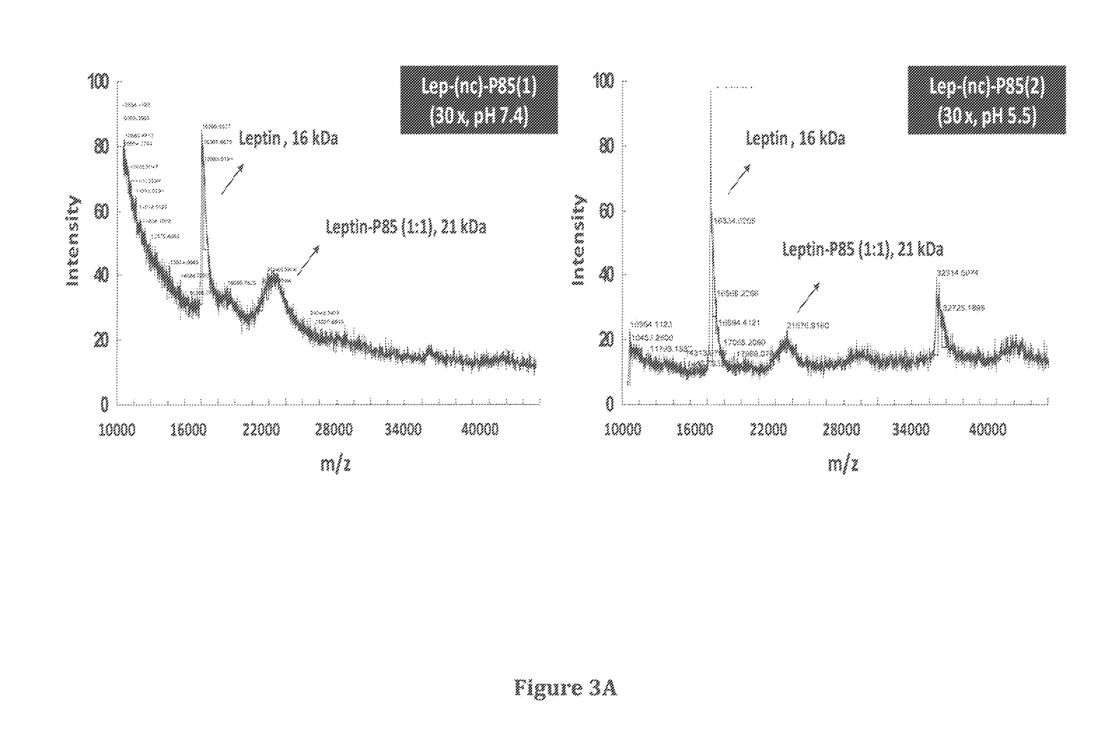

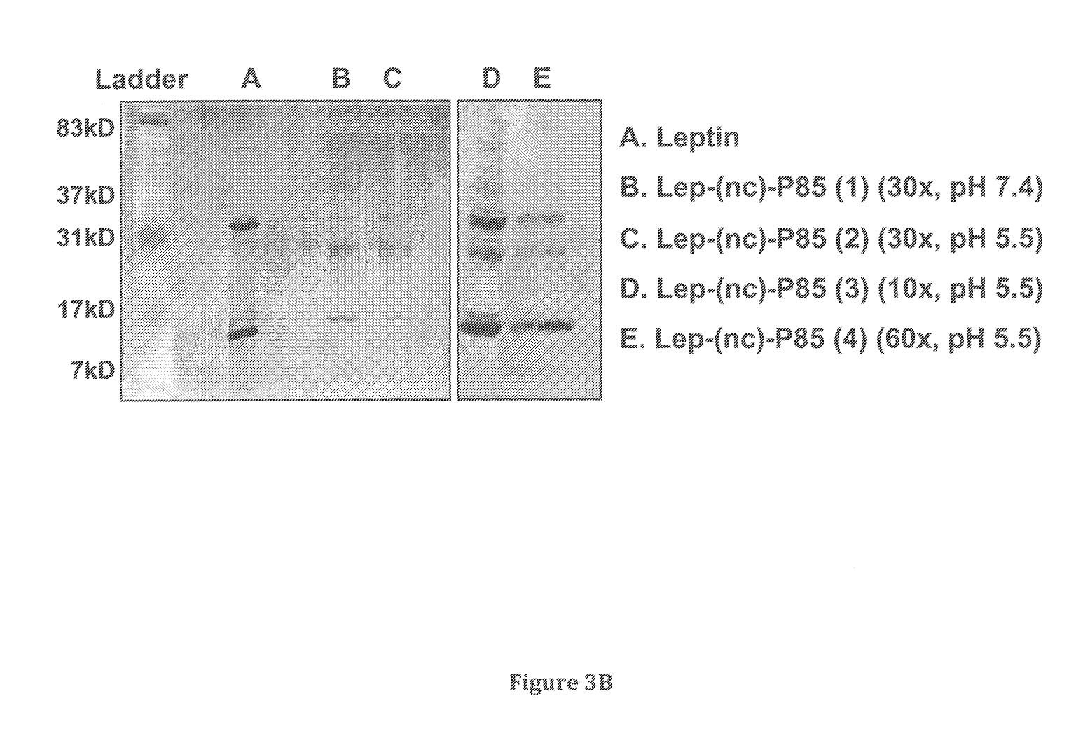

The second method involved a site-specific N-terminal modification of leptin by mono-aldehyde derivative of Pluronic® P85 (mono-aldehyde-P85) using reductive amination (FIGS. 2A and 2B). The mono-aldehyde-P85 was synthesized in a two-step procedure involving conjugation of one terminal hydroxyl group of P85 with 3-amino-1,2-propanediol and subsequent mild oxidation of the 3-amino-1,2-propanediol functionality by sodium periodate (FIG. 2B). The mono-aldehyde-P85 was immediately reacted with leptin in sodium phosphate buffer (0.1 M, pH 7.4) or sodium acetate buffer (0.1 M, pH 5.5) to produce Lep-(nc)-P85. Notably, due to the differences in pKa of α and ε amino groups (7-8 vs. 10-11) the N-terminal group of leptin was at least partially deprotonated under these pH conditions and selectively available for the reaction in contrast to the lysine groups, which were entirely protonated and not reactive. Indeed, at both pH 7.4 and pH 5.5, the products of the reaction contained a mixture of unmodified leptin and Lep-(nc)-P85 with only one P85 group attached (21 kDa), as detected by the mass spectra (FIG. 3A). This conjugate was also seen in all samples by appearance in SDS-PAGE of an additional band (ca. 21 kDa) between the unmodified leptin monomer (16 kDa) and dimer (32 kDa) (FIG. 3B). In selected samples, such as Lep-(nc)-P85(1) one can see the presence of some higher-molecular mass protein bands between ca. 37 kDa and 83 kDa that may correspond to modified dimer and larger multimeric forms. The N-terminal sequencing of the Lep-(nc)-P85 samples suggested that 60 to 70% of their N-terminal amines were blocked (presumably by P85) while the remaining 40 to 30% contained free N-terminus and represented unmodified leptin. To increase the yield of the product, different molar excesses of mono-aldehyde-P85 (10- and 60-fold) were used. However, no significant improvement in modification was achieved as shown in SDS-PAGE of the resulting conjugates, Lep-(nc)-P85(3) and Lep-(nc)-P85(4) (FIG. 3B, Lane D and E).

Purification of leptin-Pluronic® Conjugates

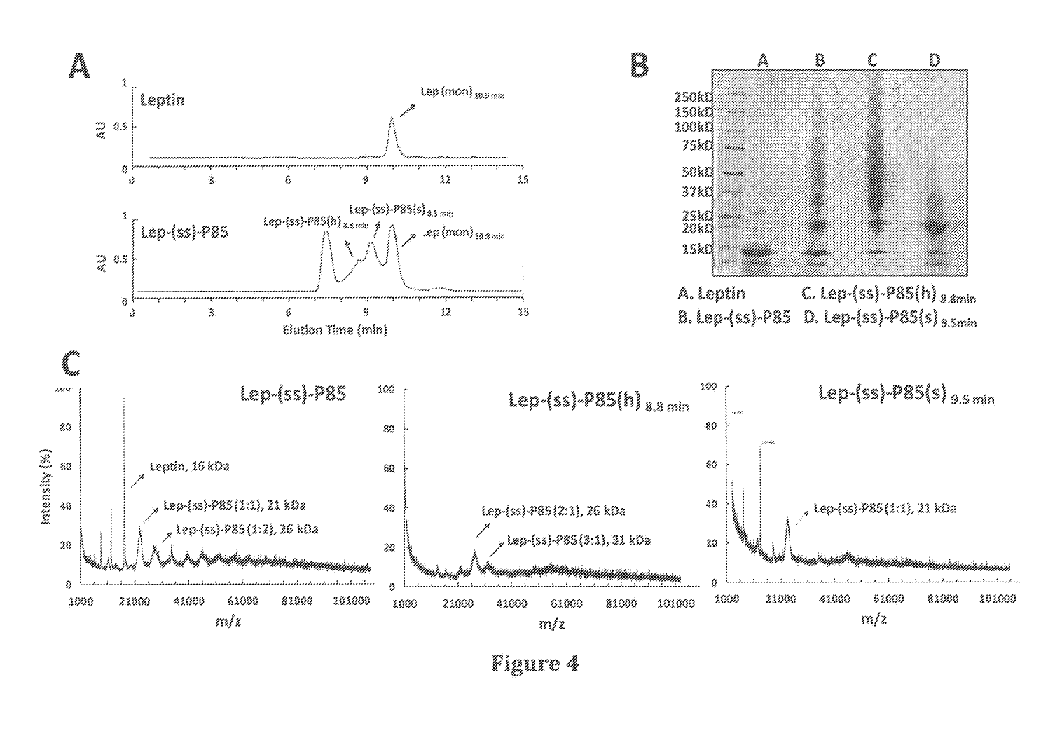

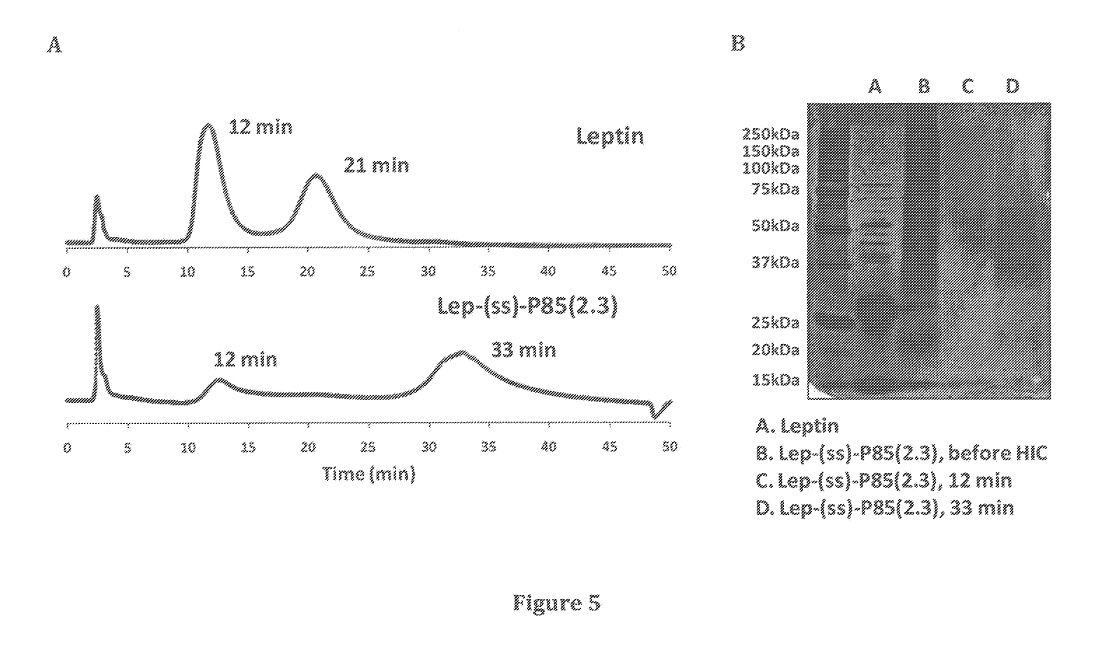

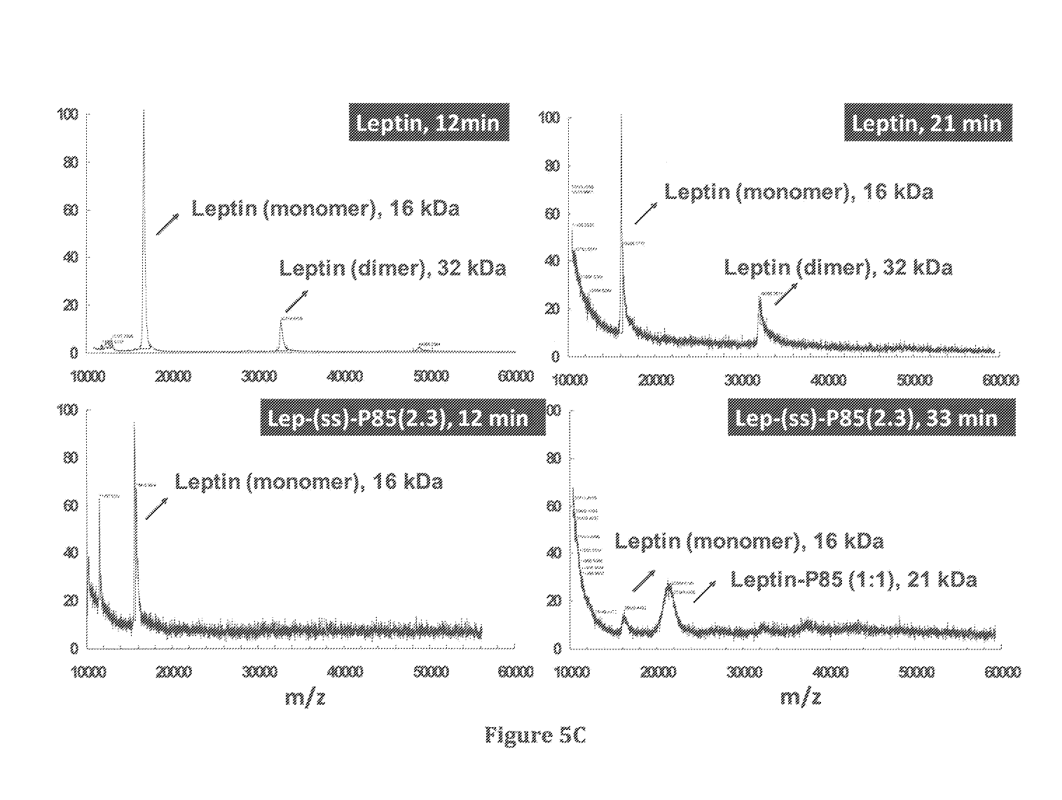

The obtained leptin-P85 conjugates (leptiPOL™) contained a mixture of unmodified leptin, leptin attached by one P85 chain (leptiPOL™-LM) and multiple P85 chains (leptiPOL™-HM). Further purification by size exclusion chromatography (SEC) was able to separate leptiPOL™-LM and leptiPOL™-HM from free leptin. SDS-PAGE and mass spectra characterized the collected fractions eluted at 9.5 min and 8 8 min to be leptiPOL™-LM and leptiPOL™-HM respectively (FIG. 4). As an alternative, HIC was performed, which allowed for the separation of the modified leptin from unmodified leptin (FIG. 5).

Immunoassays of Leptin-Pluronic® Conjugates using Anti-PEG Antibody

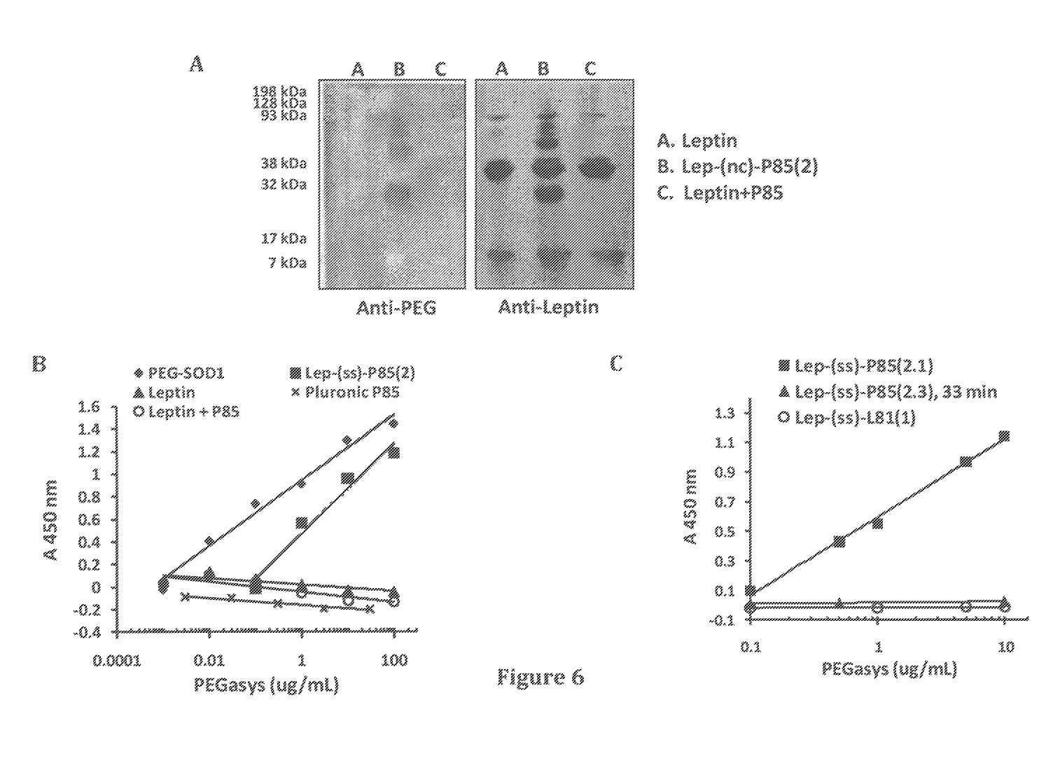

To confirm that the modified forms of leptin indeed contained Pluronic® chains, a Western blot analysis of the conjugates was performed using the monoclonal antibodies against leptin (AF498) and PEG (AGP4). The AGP4 antibodies bind to PEG backbone and can beused to assay PEGylated proteins and nanoparticles. Since Pluronic® contains PEG chains, these antibodies were used to detect the copolymer in the conjugates. As seen using Lep-(nc)-P85(2) as an example, the protein band that was ascribed above to a modified leptin monomer (21 kDa) tested positive for both leptin and PEG (contained in P85) (FIG. 6A). In addition, this method revealed a modified dimer (37 kDa) and several other modified leptin forms with high molecular mass (between 38 kDa to 93 kDa) that was stained by the antibodies but were not detected in this particular sample by either mass spectra or SDS PAGE (FIG. 3). In contrast, the native leptin or mixture of leptin and P85 reacted only with the antibody to leptin that stained the unmodified monomer (16 kDa) and dimer (32 kDa). No cross-reactions with antibodies to PEG were observed in these cases.

The presence of P85 in leptin conjugates was also confirmed by ELISA. Thus, a concentration-dependent ELISA signal was detected in both Lep-(ss)-P 85(2.1) and PEG-SOD1 (positive control) samples, but not in free P85, native leptin or P85 and leptin mixture (FIG. 6B). This indicates that 1) P85 chains became recognizable by the antibodies only after they were attached to leptin and 2) the leptin-P85 conjugates were present in the analyzed samples. Interestingly, the sensitivity of the assay also appeared to depend on the degree of modification. Thus, Lep-(ss)-P85(2.3) purified by HIC and enriched with leptin monomer conjugated to a single P85 chain exhibited much less response in ELISA than Lep-(ss)-P85(2.1) that was purified by acetone and contained leptin with multiple P85 chains (FIG. 6C). This is consistent with the report that the antibodies could not recognize lysozyme conjugates with single PEG chains as small as 2 kDa and 5 kDa. The conjugates with multiple PEG chains were readily detected. Finally, it appears that ELISA did not recognize leptin conjugates with L81, a copolymer with nearly the same length of PPG but very small ethylene glycol content compared to P85. This indicates that the AGP4 antibodies are selective to PEG, but not PPG. Altogether, the presence of P85 in modified leptin can be detected by immunoassays using anti-PEG antibody.

### Reduction of Disulfide Linkage in Leptin-Pluronic® Conjugates

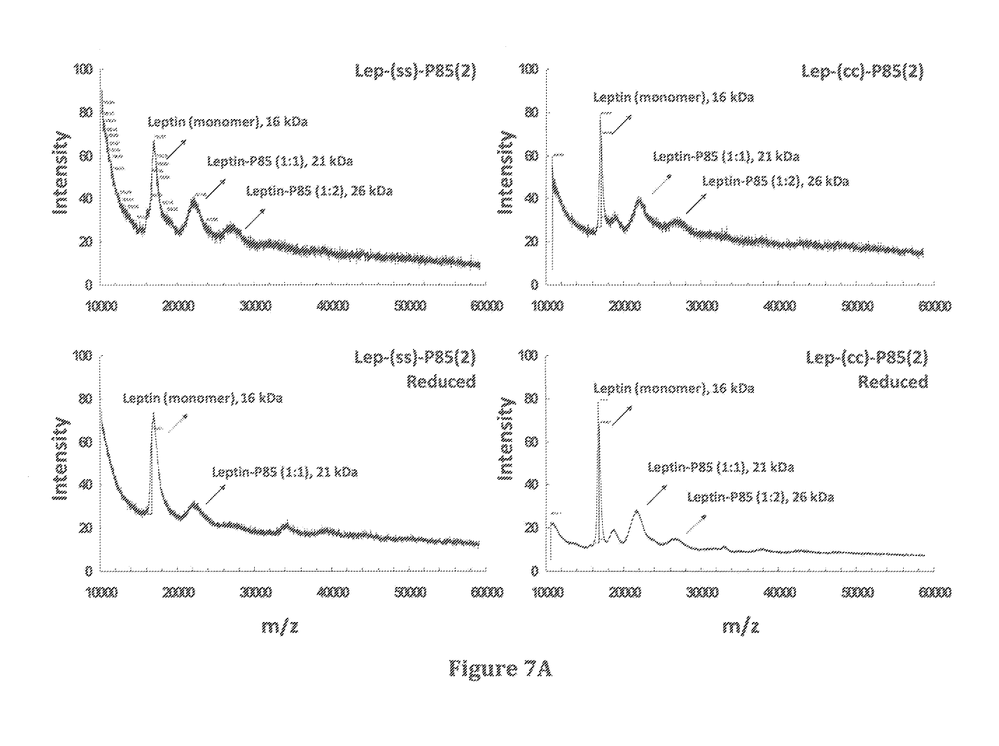

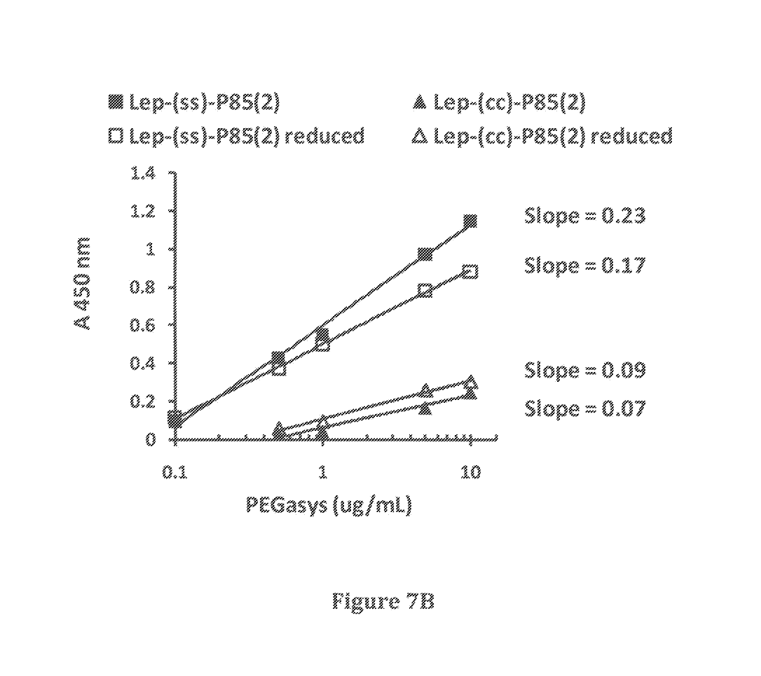

Two disulfide linked leptin-P85 samples, Lep-(ss)-P85(1) and Lep-(ss)-P85(2.1), were reduced by 6 mM L-glutathione, known to be present in the cytoplasm environment. Notably, under such reductive conditions some, but not all, P85 chains were cleaved from the protein. This was revealed in the mass spectra of the reduced Lep-(ss)-P85(2.1) by the disappearance of a signal for leptin modified with two P85 chains and a decrease of a signal for leptin modified with one P85 chain (FIG. 7A). The ELISA results also indicated a decrease in the block copolymer content in the reduced sample (FIG. 7B). In contrast, Lep-(cc)-P85(2.1), the non-degradable samples synthesized upon similar conditions as Lep-(ss)-P85(2.1), did not display any changes in the mass spectra and ELISA signal before or after treatment by L-glutathione (FIG. 7). Similarly, a partial reduction of disulfide linker was also observed for Lep-(ss)-P85(1) and was confirmed by mass spectra and ELISA, whereas no reduction was detected in the non-degradable control sample Lep-(cc)-P85(1).

### Size Measurement of Leptin-Pluronic® Conjugates

The effective diameters (Deff) measured by DLS for the native leptin, Lep-(ss)-P85(1), Lep-(ss)-P85(2.1) and HIC-purified Lep-(ss)-P85(2.3) at 100 μg/ml protein in distilled water were 3.8±0.2 nm (PDI 0.27), 15±1 nm (PDI 0.43), 7±1 nm (PDI 0.56) and 10.4±3.5 nm (PDI 0.60) respectively. The increased size and PDI indices were observed for all conjugate samples, probably, due to self-assembly of Pluronic®-modified protein.

### Binding of Leptin-Pluronic® Conjugates to the Leptin Receptor

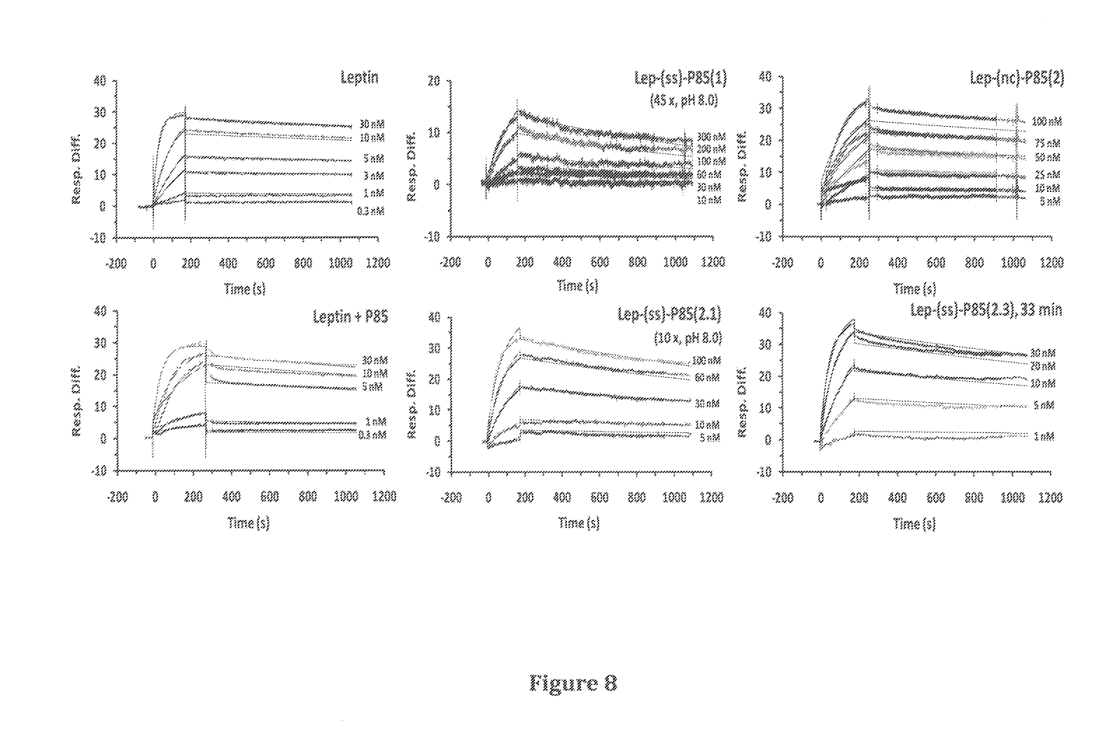

Next, the binding of the leptin-Pluronic® conjugates with the chimera leptin receptor (ObR-Fc) was studied. ObR-Fc contains the human protein G Fc fragment and the mouse ObR N-terminus that shares the same sequence as the extracellular domains of the putative leptin transporter at the BBB (ObRa) and the leptin receptor (ObRb) expressed in the brain. The Fc fragment of ObR-Fc was used to reversibly bind to protein G that was pre-immobilized onto the sensor chip. The ObR N-terminus of ObR-Fc adsorbed onto the sensor chip was used for sample detection. The SPR association and dissociation profiles were recorded for native leptin, a mixture of leptin and P85, Lep-(ss)-P85(1), Lep-(ss)-P85(2.1), Lep-(ss)-P85(2.3) (fractions purified by HIC and collected at 33 min) and Lep-(nc)-P85(2) (FIG. 8 and Table 3). In addition, for two samples that were 1) a highly modified Lep-(ss)-P85(1) and 2) a less extensively modified Lep-(ss)-P85(2.1), reductive degradation of the disulfide bond to cleave P85 chains was performed and the effects of the cleavage on the interactions of the protein with the receptor were examined The kinetic constants, ka (“on rate”), kd (“off rate”) and K_{D} (equilibrium dissociation constant) are summarized in Table 3. Native leptin displayed very fast association and extremely slow dissociation phases, resulting in a KD of ca. 10⁻¹⁰ M. Free P85 appeared to decrease the binding affinity of the native leptin by about 3 fold. The largest changes were observed for the leptin conjugates, which all displayed much slower association rates and similar dissociation rates as the native leptin. The highest K_{D} value of ca. 5.7×10⁻⁸ M was observed for the most heavily modified Lep-(ss)-P85(1), indicating that this conjugate underwent the greatest loss of affinity to ObR-Fc as a result of modification. The affinity of Lep-(ss)-P85(2.1) with its lower modification degree was at least one order of magnitude better, K_{D}=3.2×10⁻⁹ M, but still much worse than either native leptin or its mixture with P85. The affinity further improved after HIC purification, resulting in a K_{D} as low as 3.2×10⁻⁹ M for Lep-(ss)-P85(2.3) fraction (33 min) containing leptin modified with one P85 chain. The K_{D} values for the N-terminal modified Lep-(nc)-P85(2), which also had low modification degree, were similar to those observed for Lep-(ss)-P85(2.1) without HIC purification. In addition to the above, the in vitro activity measurement for mu-leptiPOL™-LM and mu-leptiPOL™-HM were 10-20 folds less active than native leptin measured as IC₅₀ in a cell proliferation assay using BaF3 mouse pro-B cells transfected with human leptin receptor.

Therefore, modification with P85 significantly decreased affinity of leptin to ObR. This decrease appeared to depend more on the extent of modification but less on the point of the copolymer attachment to leptin. For both Lep-(ss)-P85(1) and Lep-(ss)-P85(2.1) differing in modification degree the cleavage of P85 chains by 6 mM L-glutathione resulted in several fold increase in K_{D}. Notably, the restored affinity of these modified proteins did not reach the level observed with the native leptin. Altogether, the SPR data indicate that although Pluronic® P85 modifications impair the binding of leptin to its receptor, the loss of affinity can be reduced by decreasing the modification degree of the conjugates, modifying leptin N-terminus, or reduction of the disulfide linkage.

TABLE 3
Kinetic constants for leptin and leptin-P85 conjugates and leptin
receptor interaction. The association and dissociation rate
constants kₐ and k_{d} were determined as global fitting parameters
for a 1:1 binding model. The kinetics of the interaction with
leptin, the mixture of leptin and P85, Lep-(ss)-P85(1) and its
reduced form, Lep-(ss)-P85(2.1) and its reduced form,
Lep-(nc)-P85(2) and purified Lep-(ss)-P85(2.3) from HIC, 33 min
were analyzed. The equilibrium dissociation constant K_{D} was
determined as k_{d}/kₐ. Numbers represent averaged values from
three independent measurements on the same ObR-Fc surface.

| | kₐ | k_{d} | K_{D} | |
|---|---|---|---|---|
| Samples | [10⁵ M⁻¹s⁻¹] | [10⁻⁴ s⁻¹] | [10⁻¹⁰ M] | χ² |
| Leptin | 20.9 ± 1.36 | 2.08 ± 0.07 | 1.0 ± 0.03 | 0.1-0.4 |
| Leptin + P85 | 10.9 ± 0.40 | 3.68 ± 0.06 | 3.37 ± 0.08 | 0.3-0.5 |
| Lep-(ss)- | 0.12 ± 0.02 | 6.42 ± 0.03 | 571 ± 91.50 | 0.2-0.6 |
| P85(1) | | | | |
| Lep-(ss)- | 0.49 ± 0.02 | 7.9 ± 0.42 | 164 ± 15.50 | 0.2-0.3 |
| P85(1) | | | | |
| reduced* | | | | |
| Lep-(ss)- | 1.13 ± 0.07 | 3.55 ± 0.20 | 32 ± 3.86 | 0.2-0.4 |
| P85(2.1) | | | | |
| Lep-(ss)- | 3.11 ± 1.05 | 2.92 ± 0.56 | 12.1 ± 5.14 | 0.8-1.5 |
| P85(2.3), | | | | |
| 33 min | | | | |
| Lep-(ss)- | 1.95 ± 0.11 | 4.07 ± 0.41 | 20.9 ± 2.03 | 0.4-1.2 |
| P85(2.1) | | | | |
| reduced* | | | | |
| Lep-(nc)- | 0.77 ± 0.25 | 2.47 ± 0.40 | 38 ± 1.76 | 0.1-0.6 |
| P85(2) | | | | |

*The samples were prepared by dialysis of Lep-(ss)-P85(1) and Lep-(ss)-P85(2.1) against 6 mM L-glutathione in PBS containing 20% ethanol in and then purification in Amicon Ultra centrifugal filters.

### Non-Saturable Brain Uptake of Leptin Following INB Delivery

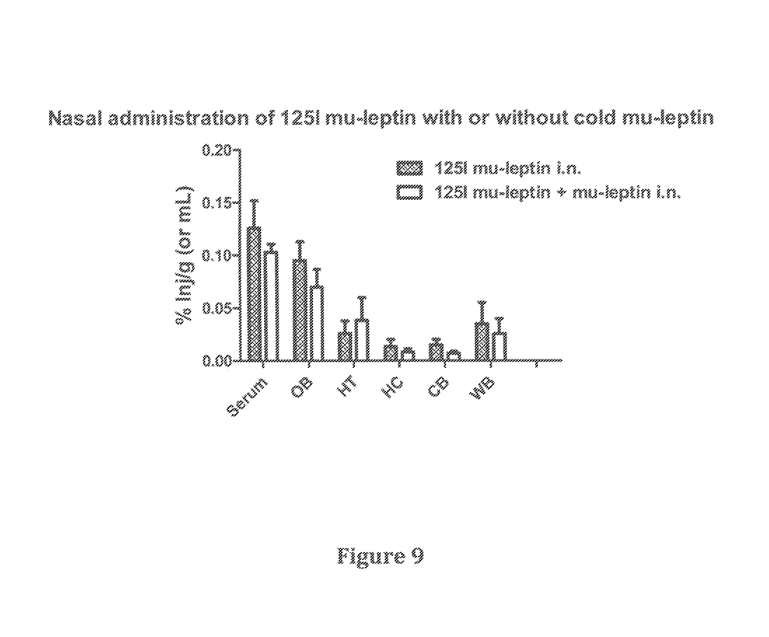

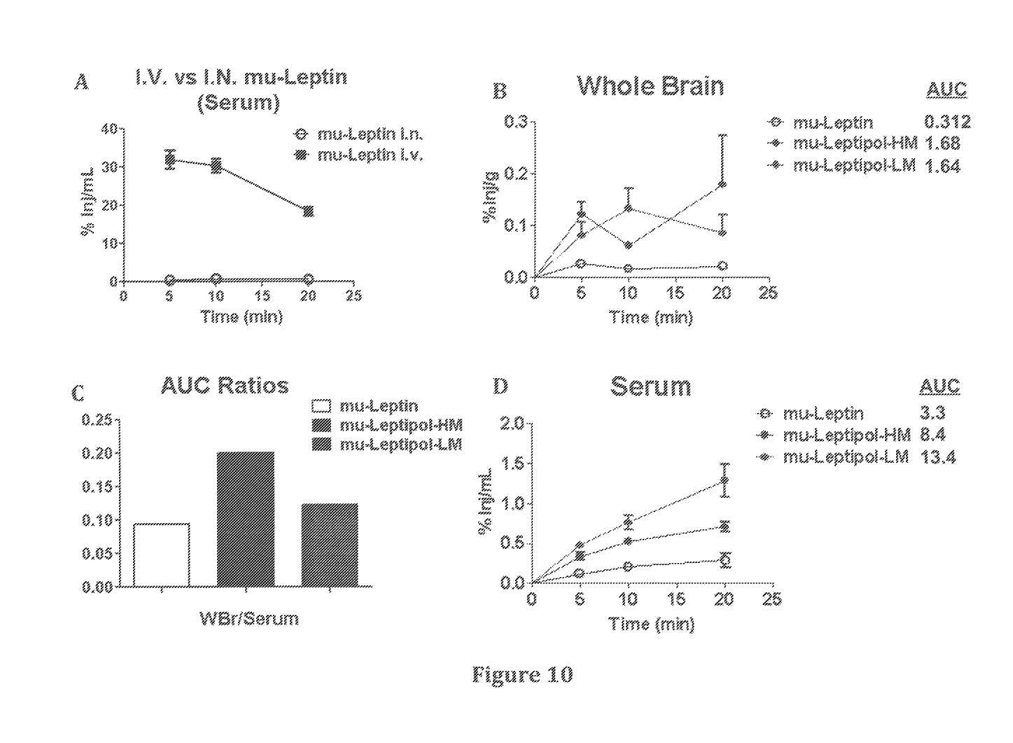

INB delivery of mu-leptin in CD-1 mice follows the traditional pattern of uptake by the olfactory bulb with lesser amounts of uptake by other brain regions (FIG. 9) with little material entering blood (FIG. 10). Not like the entry of leptin to the brain from systemic route, the role of leptin transporter in the INB delivery seems to be less relevant despite that expression of leptin receptor was identified in the nucleus of the lateral olfactory tract (Bjorbaek et al. (1998) Mol. Cell, 1:619-25). Here, a non-saturable manner of nasal leptin uptake is shown in various brain regions (olfactory bulb (OB), hypothalamus (HT), hippocampus (HC), cerebellum (CB)) (FIG. 9).

Intranasal Targeting of leptiPOL™ and Brain PK

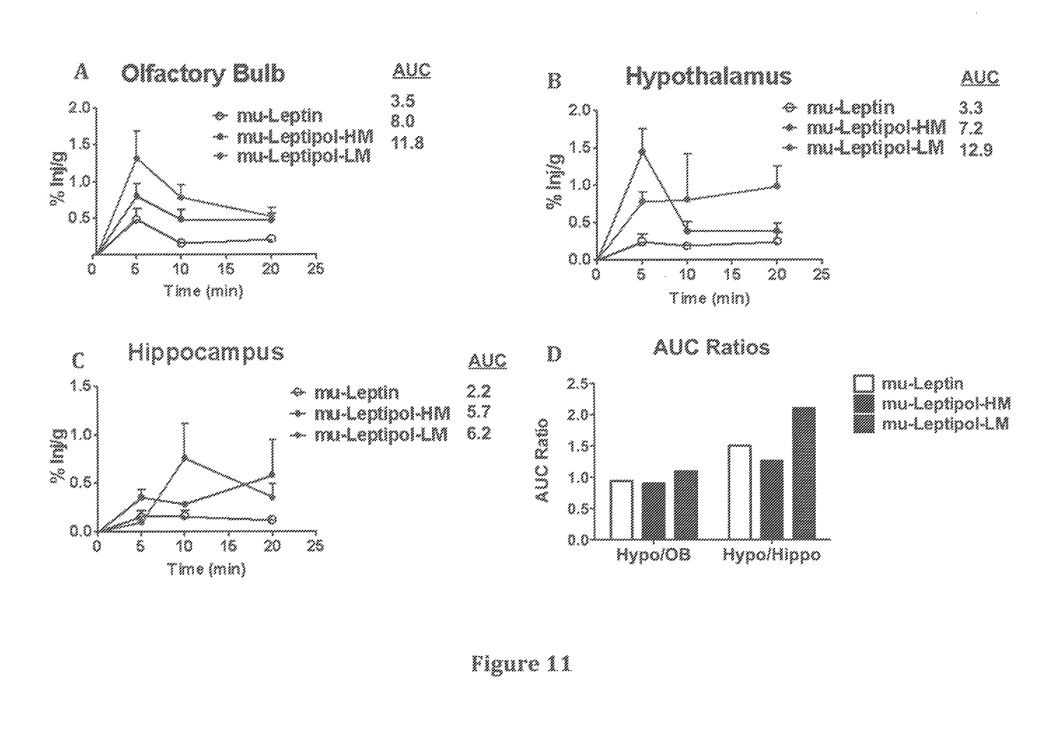

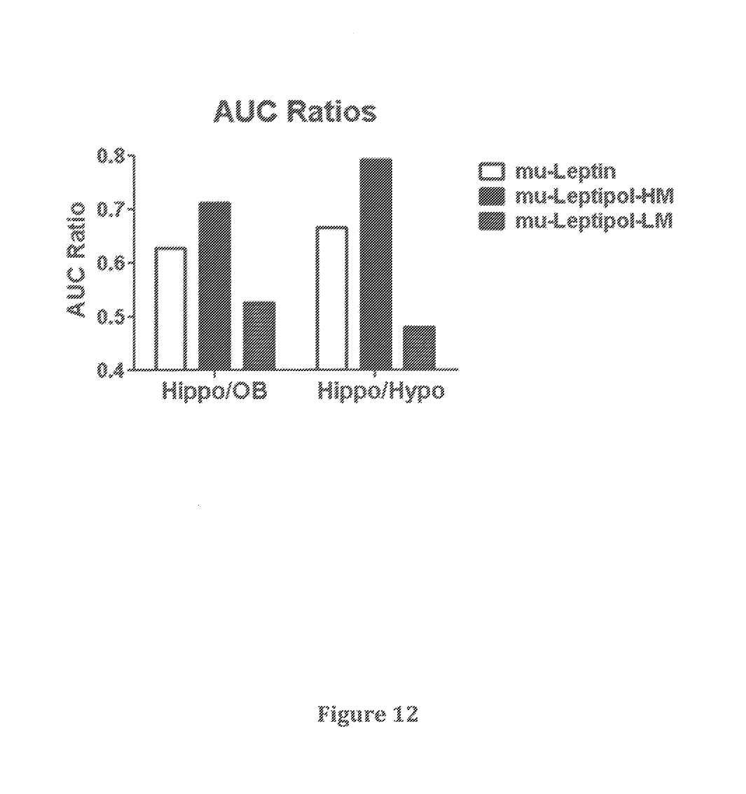

FIG. 10 shows that both leptiPOL™-LM and leptiPOL™-HM are taken up about 5 fold better by whole brain than is native mu-leptin. Entry into the blood stream is probably by way of CSF reabsorption into the blood stream (termed “bulk flow”; Dayson et al. (1996) Physiology of the CSF and Blood Brain Barriers, CRC Press, Boca Rton, Fla.) and is greater for the leptiPOL™ as well, especially the LM form (FIG. 10). Dividing the area under the curve (AUC) values for whole brain uptake by those for serum shows that the ratio is higher for the leptiPOL™. This indicates that a higher percent of material taken up by the brain is retained there rather than entering the blood stream. Thus, brain vs. periphery is relatively targeted by leptiPOL™, reducing peripheral off-target side effects such as immunogenicity. These same modifications can be used to target brain regions. As shown in the FIG. 11, uptake for the leptiPOL™ is greater than that of leptin, not just for olfactory bulb, but also for hippocampus and hypothalamus. These regions are of particular interest as these are important sites of action for leptin's effects on appetite (hypothalamus) and cognition (hippocampus). Hypothalamic uptake relative to the olfactory bulb or hippocampal uptake as assessed by AUC ratios (calculated, for example, as hypothalamus mu-leptin uptake divided by the olfactory bulb leptin uptake: 3.3/3.5=0.94) is enhanced for leptiPOL™-LM but decreased for mu-leptiPOL™-HM. In contrast, hippocampal uptake relative to olfactory bulb or hypothalamicuptake is enhanced for leptiPOL™-HM, but decreased for leptiPOL™-LM (FIG. 12). These results show that modifications with Pluronics® can enhance targeting to brain regions and depending on the modifications, different brain regions can be targeted.

### Efficacy of Intranasal LeptiPOL™ in Alzheimer's Disease Mouse Model

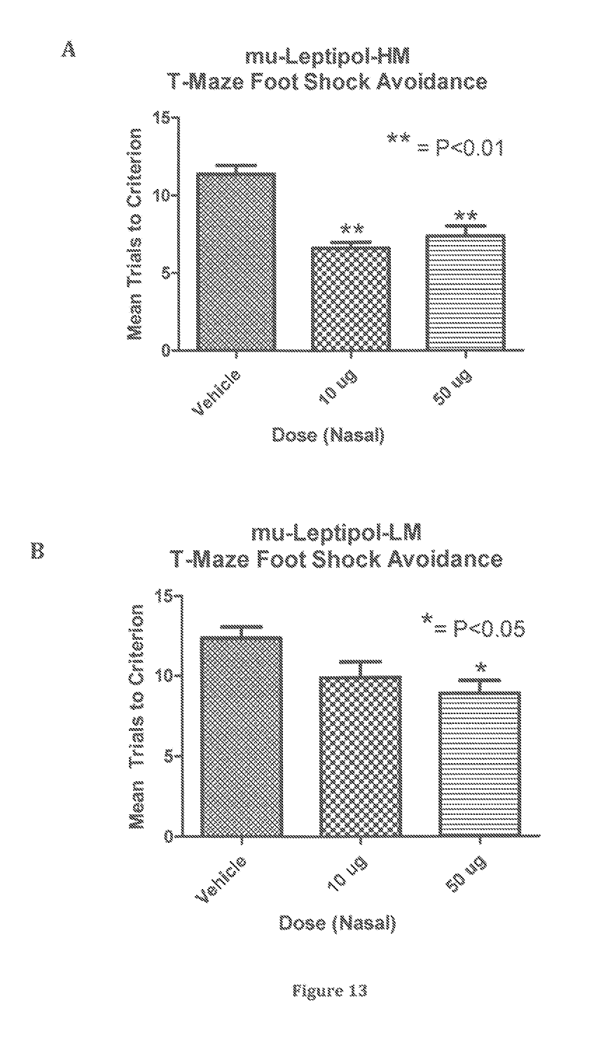

Leptin injected directly into the hippocampus is effective in reversing the cognitive impairment of 12 month old SAMP8 mice at a dose of 0.25 microg (Farr et al. (2006) Peptides, 27:1420-5). INB delivery of leptiPOL™-LM at the 50 microg dose and leptiPOL™-HM at 10 and 50 microg improved memory in the active avoidance T-maze (FIG. 13). These doses are consistent with the pharmacokinetics demonstrated here: 50 microg intranasal (FIG. 13) approximately 0.5% of the intranasal injected dose taken up by hippocampus =0.25 microg delivered to hippocampus. This indicates that leptiPOL™-HM is about 5 times more potent than leptin or leptiPOL™-LM.

Feeding Studies of Intranasal leptiPOL™

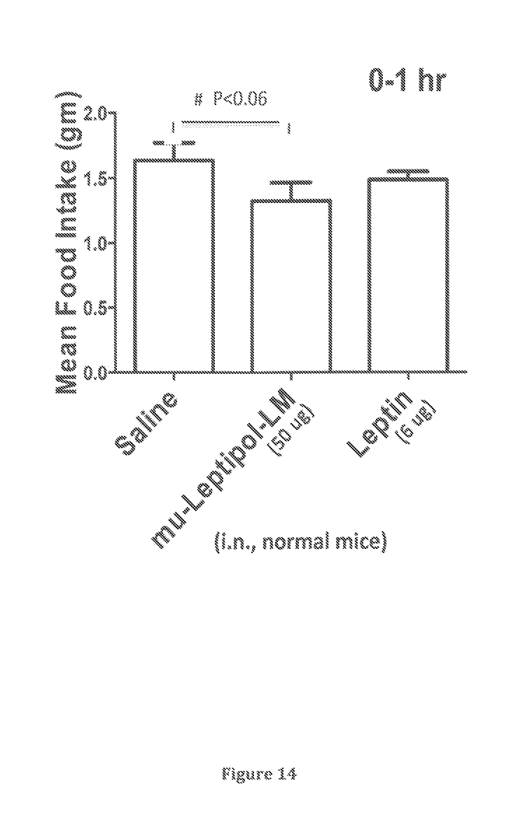

Nasal administration of leptiPOL™-LM maintained its central activity to control appetite in normal body weight mice. 50 μg of mu-leptiPOL™-LM was INB delivered to CD-1 mice that were food-deprived for 18 hours before experiment (FIG. 14). An arithmetic decrease in food intake of 19% was found for the first 1 hour. While this single experiment did not reach statistical significance (n=8/group, p=0.06 by one tailed t test), power analysis indicated that doubling the n would produce statistical significance at p<0.05.

Thus, leptiPOL™ was successfully synthesized and characterized using various analytic and bioanalytic methods. Depending on the modification degree, nasal leptiPOL™-LM shows better hypothalamus targeting than leptin or leptiPOL™-HM and is effective to reduce food intake. Nasal leptiPOL™-HM shows significant hippocampus targeting and also better efficacy to improve cognitive function. Therefore it is used for treatment of mental disorders such as AD. This work demonstrates that leptiPOL™ administration via nasal cavity can access the brain with significant amount in particular in hippocampus and hypothalamus and attain therapeutic effect.

### EXAMPLE 2

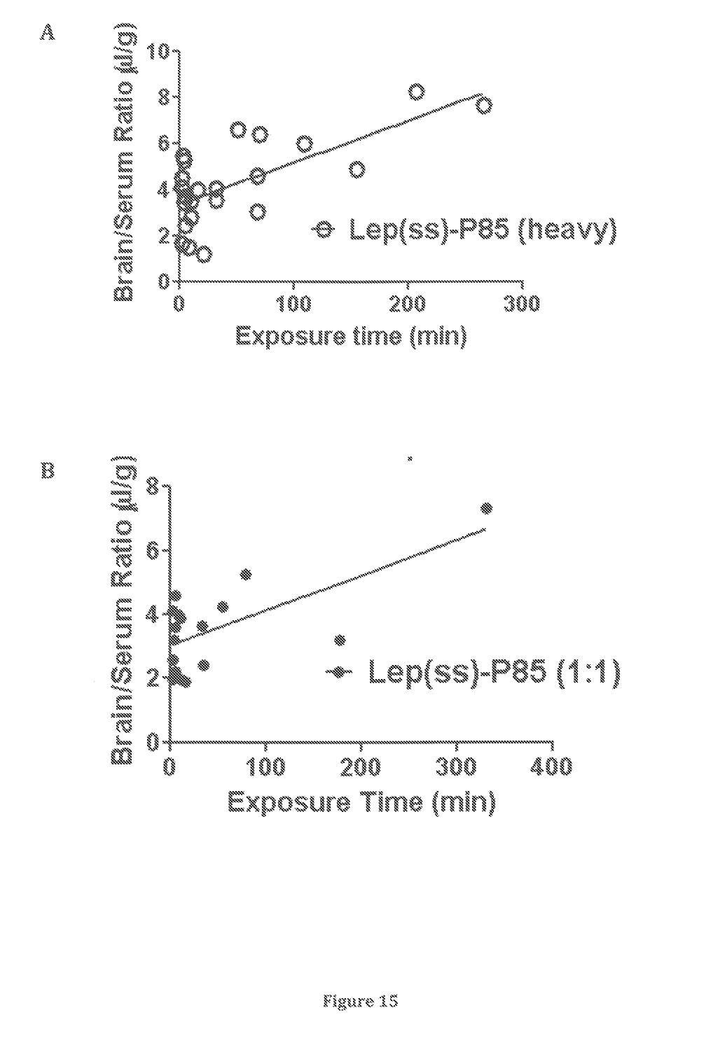

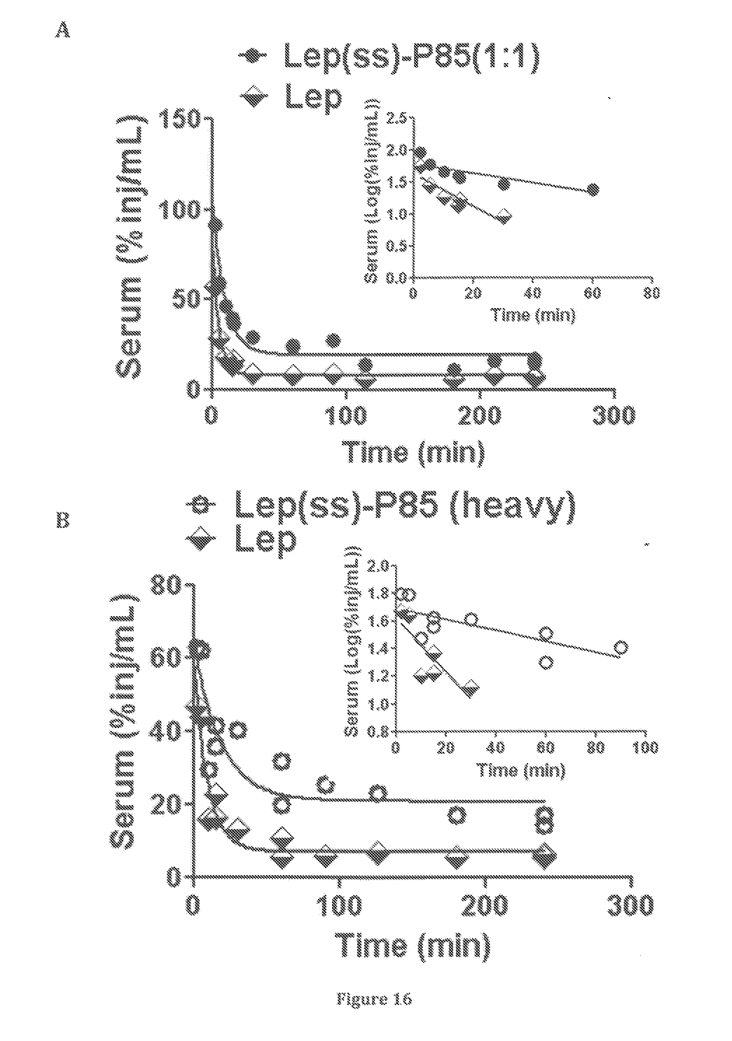

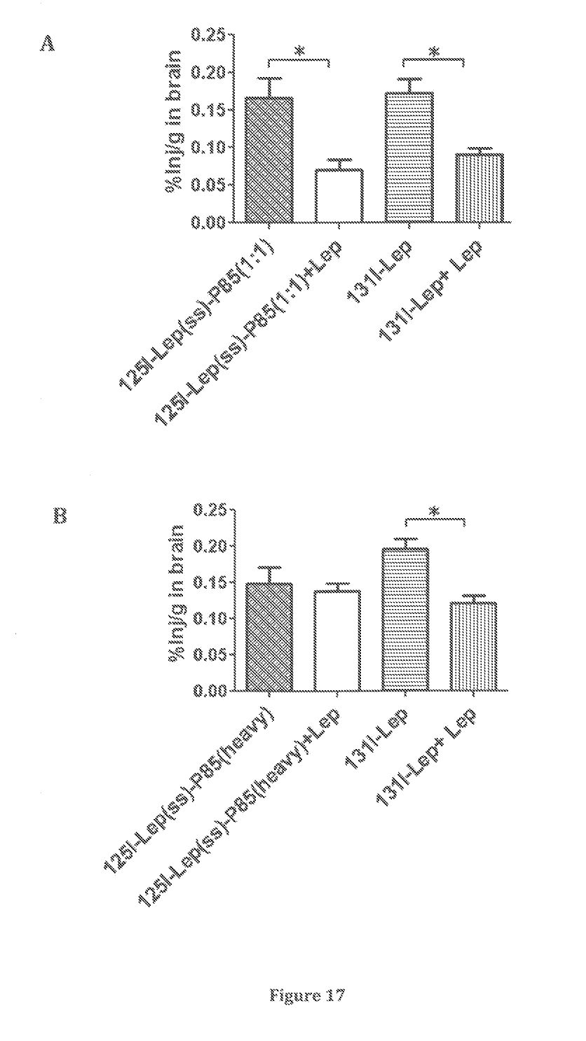

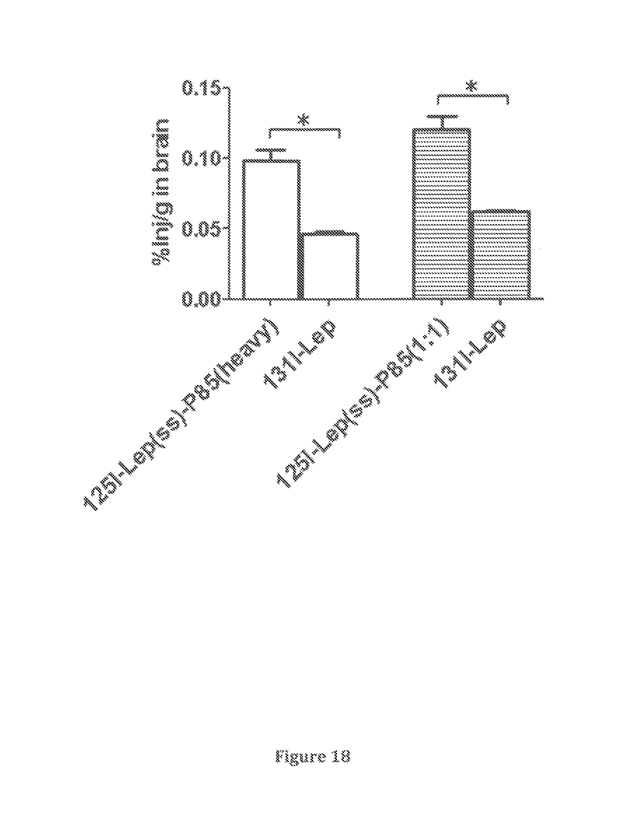

The purified leptin-P85 conjugates (lep(ss)-P85(heavy) or leptiPOL™-HM and lep(ss)-P85(1:1) or leptiPOL™-LM) were iodinated and trace amount was intravenously injected to CD-1 mice. Brain and serum samples are collected at various time points following injection and counted in gamma counter. The influx rate to cross the BBB and the serum clearance are evaluated (FIG. 15 and FIG. 16). The stability of iodinated samples in both serum and brain were measured by acidic precipitation (Tables 4 and 5). In summary, optimized leptin-Pluronic® conjugates showed longer circulation but slower entry to brain than previous generation conjugates or native leptin. Single chain modified leptin crossed the BBB via leptin transporter and heavily modified leptin crossed the BBB independent of leptin transporter. Optimized leptin-Pluronic® conjugates showed higher levels of brain accumulation in intact form than that of native leptin and are more stable than native leptin. Furthermore, the transport of Lep(ss)-P85(1:1) across the BBB is leptin transporter dependent while Lep(ss)-P85(heavy) is non-saturable and leptin transporter independent (FIG. 17). The brain uptake of intact leptin-P85 conjugates for both heavy and 1:1 form was higher than that of leptin, as shown in FIG. 18.

TABLE 4
Acid precipitation of radioactive labeled leptin analogs in brain and serum.

| Time | Leptin | | Lep(ss)-P85(1:1) | | Lep(ss)-P85(heavy) | |
|---|---|---|---|---|---|---|
| (min) | Serum(%) | Brain(%) | Serum(%) | Brain(%) | Serum(%) | Brain(%) |
| 15 | 94.41 ± 3.57 | 99.96 ± 3.93 | 104.01 ± 0.68 | 99.13 ± 1.63 | 100.25 ± 6.54 | 98.85 ± 5.64 |
| 60 | 76.21 ± 8.52 | 73.01 ± 13.72 | 93.30 ± 1.12 | 73.90 ± 1.58 | 96.08 ± 0.39 | 81.88 ± 0.40 |
| 240 | 52.85 ± 8.66 | 27.71 ± 9.52 | 71.01 ± 2.69 | 49.71 ± 3.34 | 86.04 ± 3.44 | 61.50 ± 3.34 |

TABLE 5
Acid precipitation of radioactive labeled leptin analogs in brain
and serum 4 hours after i.v. injection with brain washout.

| | Serum(%) | Brain(%) |
|---|---|---|
| Leptin | 35.75 ± 10.89 | 17.64 ± 16.05 |
| Lep(ss)-P85(1:1) | 74.04 ± 1.1 | 34.26 ± 3.29 |
| Lep(ss)-P85(heavy) | 86.96 ± 1.06 | 51.20 ± 8.16 |

### EXAMPLE 3

### Leptin-Poly(2-Oxazoline) Conjugation and Purification

Leptin-POx conjugate was synthesized similar to the procedure provided in Tong et al. (Mol Pharm. (2013) 10:360-77). Briefly, secondary amine of the piperazine-terminated POx was reacted with homofunctional linkers DSP. 13 mg of POx was reacted with a 20-fold molar excess of DSP in DMF. The mixture was supplemented with sodium borate buffer (0.1 M, pH 8.0) and reacted for 30 minutes at room temperature. The activated POx was purified by gel filtration on a Sephadex LH-20 column in dry dichloromethane and 1 mg of Leptin in sodium borate buffer (0.1 M, pH 8.0) was added. The reaction mixture was supplemented with 20% of ethanol and incubated overnight at 4° C. The resulting leptin-POx conjugate was purified by size-exclusion chromatography (SEC) on a Shimadzu high performance liquid chromatography (HPLC) system with a TSKgel® G2000SWx1 column (7.8×300 mm) from Tosoh Co. (Japan) using 0.1 M phosphate buffered saline (PBS, pH 6.8) as the mobile phase and UV detection at 220 nm. The conjugates were desalted and lyophilized for further characterization.

### Leptin-Poly(2-Oxazoline) Characterization

Molar mass of Leptin-POx was determined by matrix-assisted laser desorption/ionization time of flight mass spectroscopy (MALDI-ToF MS) using saturated sinapic acid solution in 50% acetonitrile and 0.1% aqueous TFA as the matrix. The mass spectrometer was calibrated against insulin (5729.61 Da) and albumin (66,429.09 Da).

Sodium dodecyl sulfate polyacrylamide gel electrophoresis (SDS-PAGE) analysis was performed and the gel was fixed and stained by SYPRO® Ruby solution overnight. The degree of protein modification was determined by a TNBS assay. Briefly, 10 μL of leptin or leptin-POx solutions (0.1-0.6 mg/mL) were mixed with 10 μL of TNBS solution (1.7 mM) in 80 μL of sodium borate buffer (0.1 M, pH 9.5) and incubated at 37° C. for 2 hours. The absorbance at 405 nm was measured using a microplate reader (Spectra Max® M5, MDS, CA). The degree of modification (average number of modified amino groups) was calculated according to:

$$S = 8 \times \frac{( {A}_{native} \text{/} {C}_{native} - {A}_{modified} \text{/} {C}_{modified} )}{{A}_{native} \text{/} {C}_{native}} \qquad ( 1 )$$

where Aₙₐₜᵢᵥₑ and A_{modified} are the absorbencies and Cₙₐₜᵢᵥₑ and C_{modified} are the concentrations of leptin and leptin-POx respectively. The total number of primary amino groups including lysine residues and terminal amine group of leptin is 8.

To measure the secondary structure by circular dichroism (CD), leptin or leptin-POx was dissolved in PBS buffer (pH 7.4) at 0.5 mg/mL. CD spectra were recorded between 200 and 260 nm using an Aviv CD spectrometer with a cuvette of 0.1 cm path length. Spectra were recorded in 1 nm decrements and the given spectra correspond to the average of three wavelength scans using the pure solvent as the background. The mean residue molar ellipticity [0] was calculated by:

[θ]=(θM)/(Cl) (2)

where θ is the observed ellipticity (deg), M is the mean residue molecular weight (g/mol), C is the protein concentration (g/mL) and 1 is the optical path length (cm). Binding affinity of leptin or leptin-POx with leptin receptor was determined by surface plasma resonance (SPR). Protein G was immobilized on CM5 sensor chip using following procedure: 1) inject 115 μL NHS/EDC mixed solution (1:1, v/v) to activate dextran surface; 2) inject 60 μL protein G (200 μg/mL in 10 mM sodium acetate buffer, pH 4.0) to bind with the activated sensor surface; 3) inject 75 μL of 1 M ethanolamine hydrochloride, pH 8.5 to deactivate NHS-ester and remove non-specifically bound protein. Leptin-leptin receptor binding affinity was then determined using following procedure: 1) ObR-Fc (0.5 μg/mL, 15 μL in HBS-EP, 5 μL/min flow rate) was immobilized in channel A following 1800 s washing; 2) leptin or leptin-POx (0-300 nM, 100 μL in HBS-EP, flow rate 20 μL/min) was captured and dissociated (900 s) in channel A (ObR-Fc surface) and B (protein G surface); 3) Protein G surface was regenerated by 5 μL glycine (10 mM, pH 1.5). The sensorgram in channel A was corrected by non-specific surface binding (sensorgram in channel B) and baseline draft (sensorgram of HBS-EP buffer injection in channel A) and then fitted with a 1:1 binding model as provided by BIA evaluation software. The rate constant kₐ and k_{d}, and equilibrium association constant (K_{A}) and dissociation constant (K_{D}) are determined.

### Animal Studies

Leptin or leptin-POx samples were radioactively labeled by the chloramine-T method (Tong et al., Mol Pharm. (2013) 10:360-77). Briefly, leptin or leptin-POx conjugate was incubated with 0.5 mCi Na¹²⁵I (Perkin Elmer Life Sciences, Boston, Mass.) and 10 μg of chloramine-T freshly made for 60 seconds. The mixture was purified by Illustra™ NAP™5 columns. Fractions were collected in Eppendorf tubes that were pre-coated with 50 μL of 1% BSA in lactated Ringer's solution (LR-BSA) to prevent non-specific absorbance. The radioactivities of fractions were counted in a PerkinElmer γ-counter and trichloroacetic acid (TCA) precipitation was conducted to determine the iodine association of labeled samples. Fractions containing more than 100,000 cpm/μL and in which the iodine association by TCA precipitation was more than 90% were used for the animal study. Similarly, BSA was labeled with Na131I (Perkin Elmer Life Sciences, Boston, Mass.) by chloramine-T method.

### Animal Procedures

Pharmacokinetics (PK) studies of ¹²⁵I-Leptin-POx were carried out. These studies were conducted with CD-1 male mice (8 to 10 weeks of age, Charles River Laboratories, Inc. Wilmington, Mass.). All experiments were conducted in accordance with the National Institutes of Health Guide for Care and Use of Laboratory Animals. Mice were anesthetized by i.p. injection of urethane (40%). Radiolabeled sample was prepared and injected into the jugular vein. Blood from the carotid artery was collected at various time points. Mice were immediately decapitated and the whole brain was removed and weighed. The arterial blood was centrifuged and the serum was collected. The radioactivity of tissue and serum samples was counted in a γ-counter. In some cases, a brain washout was performed before decapitating and the brain was collected. Briefly, after opening the abdomen, arterial blood was collected from the abdominal aorta. The thorax was then opened to expose the heart. The descending aorta was clamped, both jugular veins severed, and LR-BSA was perfused over 1 minute into the left ventricle of the heart. Finally, the mouse was decapitated and the whole brain removed and weighed for further experiments.

### Serum Clearance and Influx Rate Across the Blood-Brain Barrier (BBB)

Mice anesthetized with urethane received an i.v. injection of ¹²⁵I-Leptin or ¹²⁵I-Leptin-POx with ¹³¹I-albumin (300,000 cpm of each) into the jugular vein. Blood from the carotid artery and brain was collected at various time points between 2 and 240 minutes after injection. The radioactivities of brain and serum samples were counted in a γ-counter. The percent of the i.v. injected dose ID/μL in serum (% ID/μL) and the dose taken up per gram of brain at time t (% ID/g brain) were by:

$$\begin{matrix}\% ID \text{/} μ L = \frac{{C}_{p} ( t )}{ID} \times 100 , \\ and & ( 3 ) \\ \% ID \text{/} g brain = ( \frac{{A}_{m}}{{C}_{p} ( t )} - {V}_{i ( 0 )} ) \times \frac{{C}_{p} ( t )}{ID} \times 100 & ( 4 )\end{matrix}$$

where ID is the cpm i.v. injected. Aₘ and Cₚ(t) are the cpm/g of brain and the cpm/μL of serum at time t, respectively.

The serum concentration (percent of the i.v. injected dose ID/μL in serum, % ID/μL) was plotted against time to describe the serum clearance. The slope between log(% ID/μL) and time was used to calculate the half time clearance from blood. Multiple-time regression analysis was applied to calculate the blood-to-brain unidirectional influx rate (Ki) of the radiolabeled compounds into the brain. The brain/serum ratios (μL/g) were plotted against exposure time estimated from:

Aₘ/Cₚ(t)=Kᵢ×[∫₀^{t}C_{F}(t)dt]/Cₚ(t)+Vᵢ₍ₒ₎ (5)

where Aₘ and Cₚ(t) are the cpm/g of brain and the cpm/μL of serum at time t, respectively. Ki was measured as the slope for the linear portion of the relation between brain/serum ratios and respective exposure times. The exposure time was calculated as the area under the serum concentration time curve divided by serum concentration at time t. The y-intercept of the line represents Vi (0), the distribution volume in the brain at t=0.
Brain Region Distribution of Intranasal Delivery of leptin-POx

The experiment was performed using the same method as described in Example 1 for intransal delivery of leptin-Pluronic® conjugates. The data were collected and analyzed as described in Example 1.

### Results

### Synthesis and Characterization of Leptin-POx Conjugate

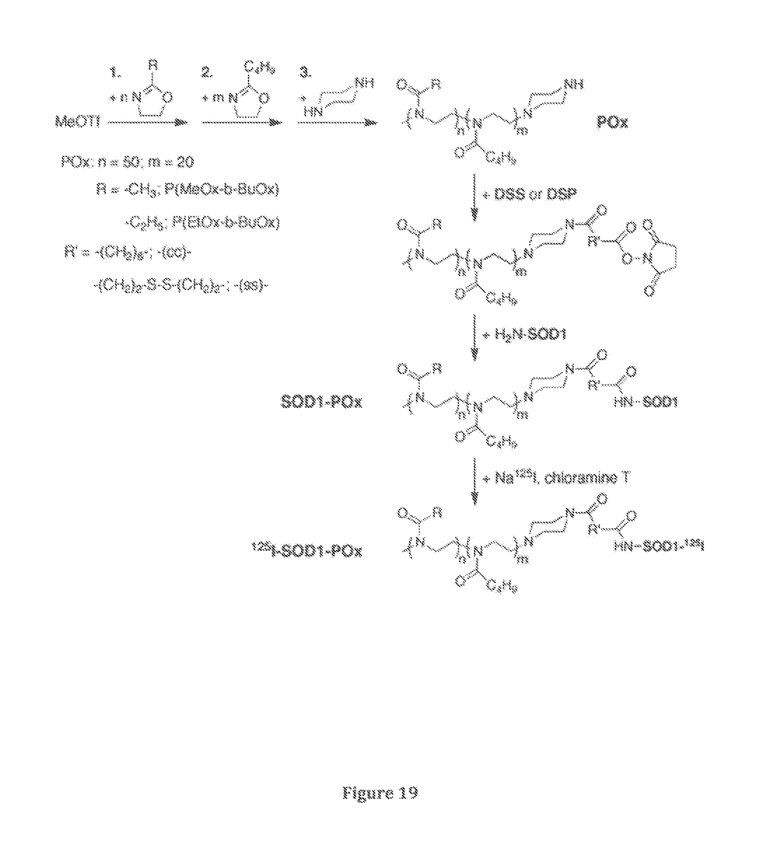

P(MeOx-b-BuOx) was selected and conjugated with leptin because it causes less aggregation and precipitation of leptin as compared to more hydrophobic P(EtOx-b-BuOx). The structure and molecular characteristics of P(MeOx-b-BuOx) were summarized in Table 6. The same two-step synthesis route was used to prepare leptin-POx conjugate (FIG. 19). Low yield of leptin-POx conjugate was observed when the second step of conjugation was carried out in aqueous buffer (pH 8.0). Therefore, 20% of ethanol was added to disrupt micelle formed with POx and improve the conjugation yield. The conjugates were purified by SEC-HPLC to remove non-modified proteins and excess of polymer. The yield of the leptin-POx conjugation varied from 50% to 60% as per initial leptin.

TABLE 6
Molecular characteristics of synthesized POx block copolymers.

| Polymer | Mₙ (×10³)^{a} | M_{w}(×10³)^{b} | Mₙ(×10³)^{b} | D^{b} | Yield(%)^{c} |
|---|---|---|---|---|---|
| P(EtOx₅₀-b- | 7.6 | 10.8 | 9.7 | 1.11 | 59 |
| BuOx₂₀) | | | | | |
| P(MeOx₅₀-b- | 6.8 | 10.2 | 8.2 | 1.25 | 89 |
| BuOx₂₀) | | | | | |

^{a}Determined by end group analysis based on 1H NMR spectroscopy data.
^{b}Determined by GPC.
^{c}Recovered yield.

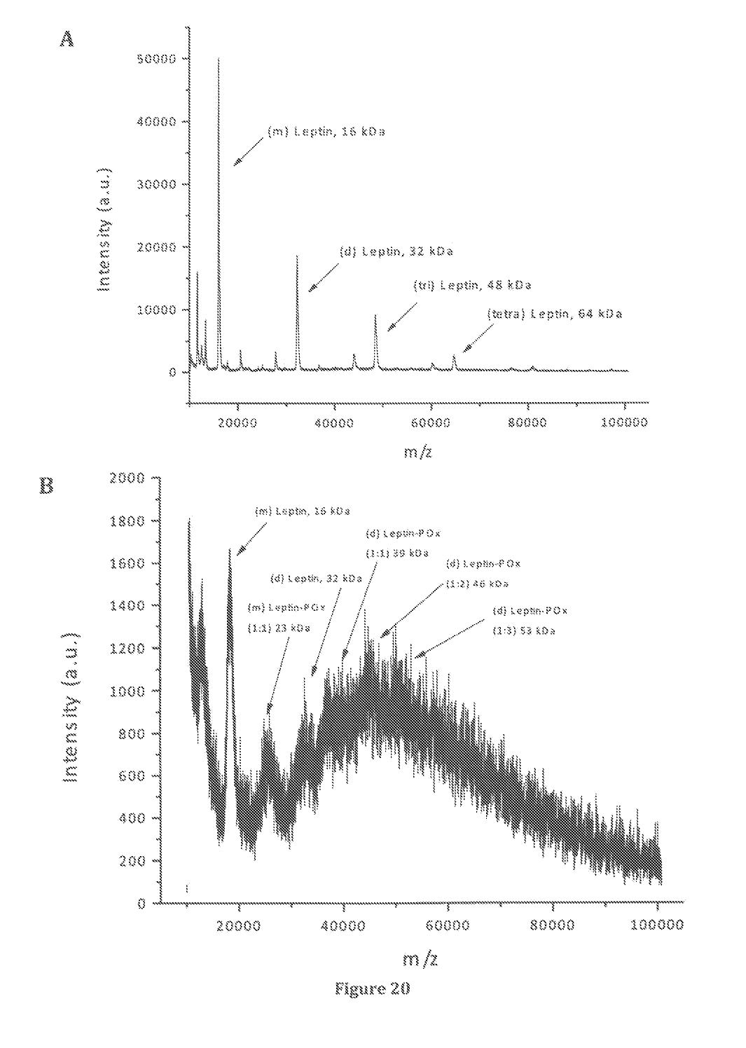

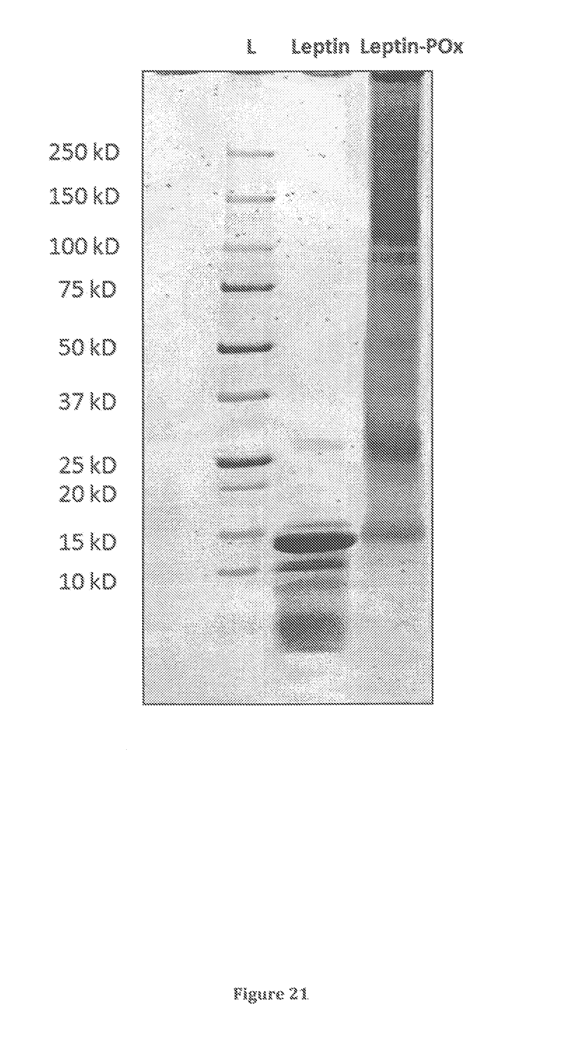

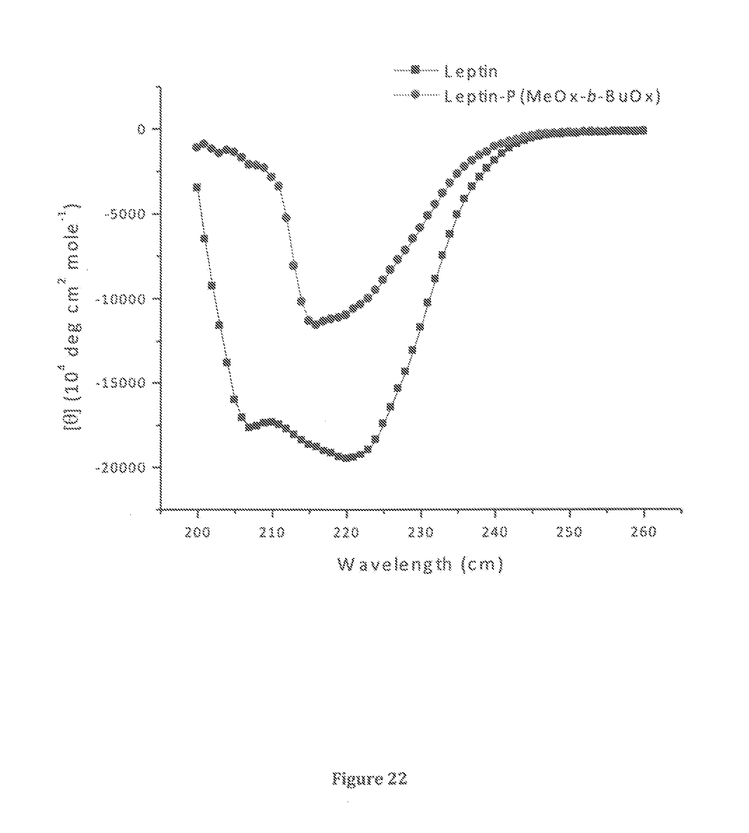

The leptin-POx conjugate was analyzed by MALDI-ToF MS, SDS-PAGE, TNBS assay, CD and SPR. The MALDI-ToF mass spectra (FIG. 20) shows that similar to SOD1, the native Leptin also contained a mixture of a protein monomer (16 kDa) and a dimer (32 kDa), and the Leptin-POx conjugates contained a mixture of a monomer with one (23 kDa) and a dimer with one (39 kDa), two (46 kDa) or three (53 kDa) polymer chains attached. In addition the conjugate samples also contained some unmodified Leptin monomer and dimer. SDS-PAGE further confirmed the existence of multiple conjugate forms in leptin-POx samples, including multiple bands with high molar mass as well as some free leptin monomer and dimer (FIG. 21). Mean modification degree of this conjugate is as determined by TNBS amino group titration assay. CD spectra (FIG. 22) showed that there is a significant decrease in CD signal for leptin-POx conjugate as compared to native leptin, indicating that a-helix component of Leptin decreased after modification. Similar result has also been observed for HRP-POx conjugate (Tong et al. (2010) Mol. Pharm.,7:984-92). SPR study showed that POx modification has significant effect on the binding affinity of leptin with leptin receptor. The equilibrium association constant (K_{A}) decreased from 1.1×10⁹ M⁻¹ for native leptin to 1.8×10⁷ M⁻¹ for leptin-POx conjugate (Table 7). However, the binding affinity of leptin-POx will be partially recovered after attached POx chains are release from protein in vivo since similar results have been observed with leptin-Pluronic® conjugate.

TABLE 7
Molecular characteristics of leptin-POx conjugate.

| | | | | | Equili- | |
|---|---|---|---|---|---|---|
| | | | | | brium | Equili- |
| | | | Rate | Rate | associa- | brium |
| | | | con- | con- | tion | dissocia- |
| | | Modifi- | stant^{b} | stant^{b} | constant^{b} | tion |
| | | cation | (kₐ) | (k_{d}) | (K_{A}) | constant^{b} |
| | Linker | degree^{a} | [M⁻¹s⁻¹] | [s⁻¹] | [M⁻¹] | (K_{D}) [M] |
| Leptin | N/A | N/A | 5.3 × | 5.1 × | 1.1 × 10⁹ | 9.6 × 10⁻¹⁰ |
| | | | 10⁵ | 10⁻⁴ | | |
| Leptin- | DSP | 6.0 | 1.3 × | 7.3 × | 1.8 × 10⁷ | 5.6 × 10⁻⁸ |
| POx | | | 10⁴ | 10⁻⁴ | | |

^{a}Counting per leptin monomer as determined by TNBS assay.^{b}Determined by SPR.

Pharmacokinetics and Brain Uptake of 1251-Leptin-Pox from Intravenous Administration

The elimination half-time of ¹³¹I albumin was determined to be 3.86 hours (leptin-POx group) or 2.85 hours (leptin group) which indicates the successful estimatation of the pharmacokinetic profiles of ¹²⁵I labeled sample and ¹³¹I-albumin in these animals (Shinoda et al. (1998) J. Pharm. Sci., 87:1521-6; Katsumi et al. (2005) J. Pharmacol. Exp. Ther., 314:1117-24). The calculated half-time disappearance of Leptin-POx was about 1.63 times longer as compared to the native protein (31.2 vs 19.2 minutes), indicating a slower elimination and increased circulation stability of this conjugate. This effect has also been observed for SOD1-POx conjugate.

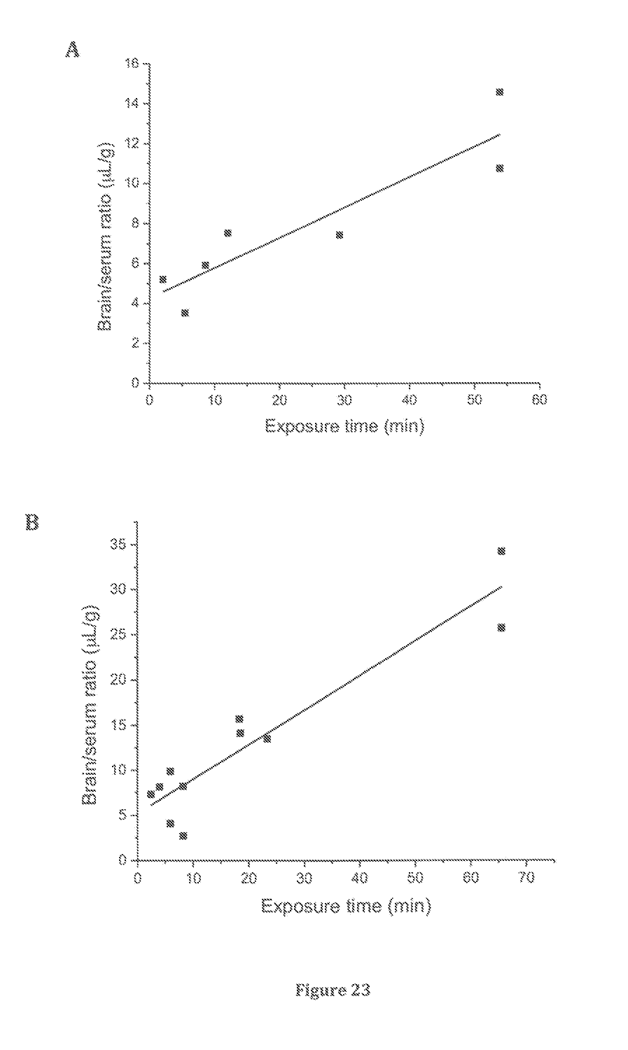

In FIGS. 23A and 23B, the brain/serum ratio of labeled native leptin and leptin-POx, corrected by the brain/serum ratio for the co-injected ¹³¹I-albumin, is plotted against exposure time to calculate the blood-to-brain influx rate. The slopes, Ki, of the linear portion (0-60 minutes) of the albumin-corrected plots, were Ki=0.151±0.031 μL/g·min (r=0.80, p <0.005; n=1-2 mice/time point) for ¹²⁵I-leptin and Ki=0.382±0.047 μL/g·min (r=0.87, p <0.0001; n=1-2 mice/time point) for ¹²⁵I-leptin-POx, demonstrating that both leptin and leptin-POx crossed the BBB significantly faster than albumin and leptin-POx showed a higher influx rate to the brain than leptin. The initial volumes of distribution in brain for leptin and leptin-POx were 4.273±0.960 μL/g and 5.203±1.407 μL/g respectively. It is well known that native leptin can cross the BBB and reach the brain due to the leptin transporter system expressed on the BBB. Here, leptin-POx conjugate also showed the capability to cross the BBB and the influx rate was higher than native leptin. This result indicates that other Leptin transporter-independent mechanism may be employed by leptin-POx to cross the BBB. Similar result has been observed by us for leptin-Pluronic® conjugate, which transported across the BBB in a non-saturated manner as administration of excess of unlabeled leptin-Pluronic® or leptin had no effect on the brain entry of radiolabeled leptin-Pluronic® (Price et al. (2010) J. Pharmacol. Exp. Ther., 333:253-63; Banks et al. (2011) Physiol. Behay., 105:145-9). This property is highly desirable because the BBB transport of leptin-Pluronic® would not be affected by leptin peripheral resistance and transporter impairment developed in obesity condition.

Brain Region Distribution and Uptake of ¹²⁵I-Leptin-POx from Nasal Administration

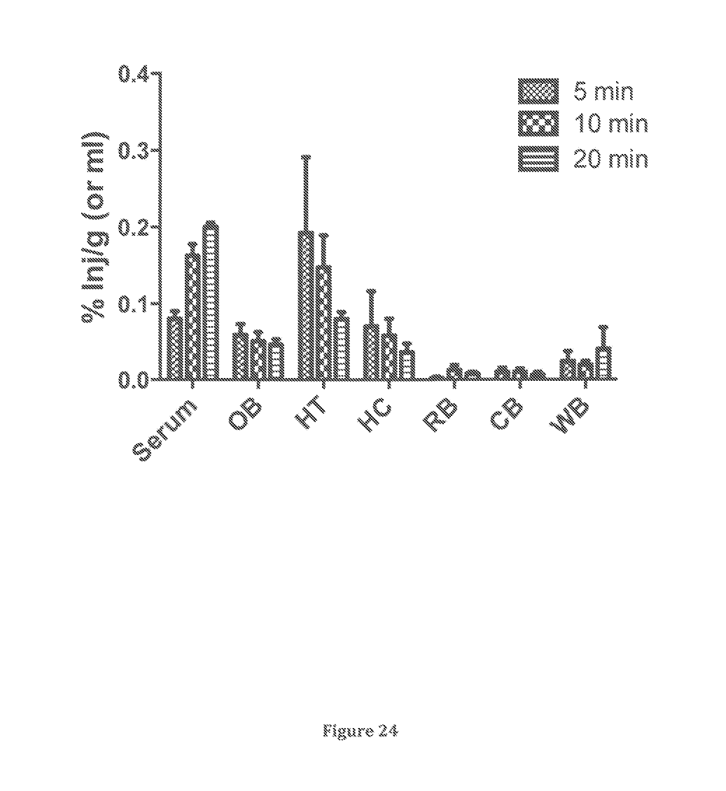

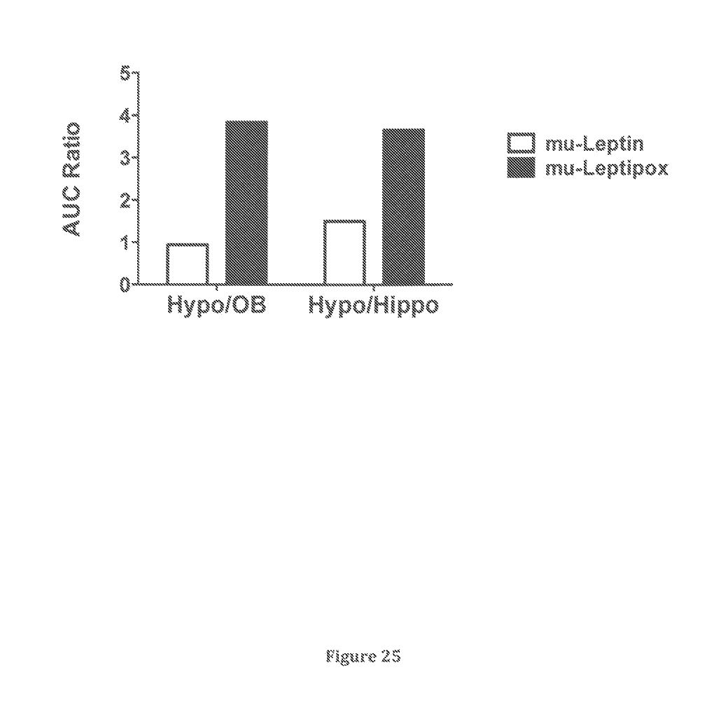

Nasal leptin-POx showed significant amount of uptake in hypothalamus, followed by hippocampus, and a lesser amount in olfactory bulb and other brain regions (FIG. 24). Importantly, the brain hypothalamus targeting relatively to olfactory bulb or hippocampus increased 4 folds or 2 folds respectively in animals receiving nasal leptin-POx comparing to animals treated with nasal leptin (FIG. 25).

Leptin-POx (P(MeOx-b-BuOx)) conjugate was synthesized using a well-established conjugation procedure. Leptin-POx was characterized with analytical techniques including mass spectroscopy, electrophoresis, TNBS assay, CD spectroscopy and SPR. This conjugate contained a mixture of proteins with different numbers of POx attached and partially maintained the conformation and receptor-binding affinity of Leptin. Animal study revealed that Leptin-POx had longer circulation half life (31.2 vs 19.2 min) and increased influx rate to the brain (0.382±0.047 μL/g·min vs 0.151±0.031 μL/g·min) than those of native Leptin. Nasal leptin-POx access to the brain via nasal route with improvement in targeting to hypothalamus than leptin.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for inhibiting or treating obesity in a subject, said method comprising intranasally administering to said subject a composition comprising a therapeutic protein conjugated to one amphiphilic block polymer.

2. The method of claim 1, wherein said therapeutic protein is selected from the group consisting of leptin, glucagon-like peptide 1 (GLP-1), oxyntomodulin (OXM), peptidy YY (PYY), ghrelin, pancreatic polypeptide, and amylin.

3. The method of claim 2, wherein said therapeutic protein is leptin.

4. The method of claim 1, wherein said therapeutic protein is conjugated to the amphiphilic block copolymer via a linker.

5. The method of claim 1, wherein said amphiphilic block copolymer is a copolymer comprising at least one hydrophilic poly(2-oxazoline) segment and at least one hydrophobic poly(2-oxazoline) segment.

6. The method of claim 1, wherein said amphiphilic block copolymer is a copolymer comprising at least one poly(oxyethylene) segment and at least one poly(oxypropylene) segment.

7. The method of claim 1, wherein said amphiphilic block copolymer is a copolymer comprising at least one poly(oxyethylene) segment and at least one segment selected from the group of polylactic acid (PLA), poly(lactide-co-glycolide) (PLG), poly(lactic-co-glycolic acid) (PLGA), and polycaprolactone (PCL).

8. The method of claim 1, wherein said composition is substantially void of therapeutic protein conjugated to more than one amphiphilic block polymer.

9. A method for inhibiting or treating dementia in a subject, said method comprising intranasally administering to said subject a composition comprising a therapeutic protein conjugated to more than one amphiphilic block polymer.

10. The method of claim 9, wherein said subject has Alzheimer's disease.

11. The method of claim 9, wherein said therapeutic protein is leptin.

12. The method of claim 9, wherein said therapeutic protein is conjugated to the amphiphilic block copolymer via a linker.

13. The method of claim 9, wherein said amphiphilic block copolymer is a copolymer comprising at least one hydrophilic poly(2-oxazoline) segment and at least one hydrophobic poly(2-oxazoline) segment.

14. The method of claim 9, wherein said amphiphilic block copolymer is a copolymer comprising at least one poly(oxyethylene) segment and at least one poly(oxypropylene) segment.

15. The method of claim 9, wherein said composition is substantially void of therapeutic protein conjugated to one amphiphilic block polymer.

16. The method of claim 9, wherein said amphiphilic block copolymer is a copolymer comprising at least one poly(oxyethylene) segment and at least one segment selected from the group of polylactic acid (PLA), poly(lactide-co-glycolide) (PLG), poly(lactic-co-glycolic acid) (PLGA), and polycaprolactone (PCL).

17. A method for inhibiting or treating obesity in a subject, said method comprising intravenously administering to said subject a composition comprising a therapeutic protein conjugated to more than one amphiphilic block polymer.

18. The method of claim 17, wherein said therapeutic protein is selected from the group consisting of leptin, glucagon-like peptide 1 (GLP-1), oxyntomodulin (OXM), peptidy YY (PYY), ghrelin, pancreatic polypeptide, and amylin.

19. The method of claim 18, wherein said therapeutic protein is leptin.

20. The method of claim 17, wherein said therapeutic protein is conjugated to the amphiphilic block copolymer via a linker.

21. The method of claim 17, wherein said amphiphilic block copolymer is a copolymer comprising at least one hydrophilic poly(2-oxazoline) segment and at least one hydrophobic poly(2-oxazoline) segment.

22. The method of claim 17, wherein said amphiphilic block copolymer is a copolymer comprising at least one poly(oxyethylene) segment and at least one poly(oxypropylene) segment.

23. The method of claim 17, wherein said composition is substantially void of therapeutic protein conjugated to one amphiphilic block polymer.

24. The method of claim 17, wherein said amphiphilic block copolymer is a copolymer comprising at least one poly(oxyethylene) segment and at least one segment selected from the group of polylactic acid (PLA), poly(lactide-co-glycolide) (PLG), poly(lactic-co-glycolic acid) (PLGA), and polycaprolactone (PCL).
